(19) United States
(12) Patent Application Publication
(10) Pub. No.: US 2015/0164761 A1
(43) Pub. Date: Jun. 18, 2015

(54) HAIR TREATMENT COMPOSITIONS COMPRISING SELECTED FATTY ACID AMIDES AND SELECTED FRAGRANCES

(71) Applicant: Henkel AG & Co. KGaA, Duesseldorf (DE)

(72) Inventor: Marcus Kruger, Ellerhoop (DE)

(21) Appl. No.: 14/614,627

(22) Filed: Feb. 5, 2015

Related U.S. Application Data
(63) Continuation of application No. PCT/EP2013/065009, filed on Jul. 16, 2013.

(30) Foreign Application Priority Data
Aug. 8, 2012 (DE) 10 2012 214 042.5

(51) Int. Cl. A61K 8/49; A61Q 5/12; A61Q 5/02

(52) U.S. Cl. CPC A61K 8/4946; A61Q 5/02; A61Q 5/12; A61K 2800/59

(57) ABSTRACT

The present invention relates to hair treatment compositions comprising a synergistic combination of selected fatty acid amides and further selected fragrances.

### FIELD OF THE INVENTION

The present invention generally relates to hair treatment agents containing a synergistic combination of selected fatty acid amides and further selected fragrances.

### BACKGROUND OF THE INVENTION

The need exists to improve haircare products further and to confer further advantageous properties on them. In particular, a care complex should be provided which, ideally, can also be employed in conjunction with oxidizing agents and surfactant agents.

The consumer frequently complains not of inadequate combability of keratinic fibers but rather about the scent impression of washed keratinic fibers, in particular hours after washing and styling when the keratinic fibers have been exposed to the environment for a prolonged period. The odor of the keratinic fibers fades or changes markedly when the washed keratinic fibers are exposed in the environment to influences such as smog, smoke, in particular smoke from tobacco products, greasy smells in kitchens or excessively perfumed premises such as department stores etc. Here, the consumer increasingly expects a hair treatment agent which still smells clean even hours after washing and styling and spending time in such an environment.

Environmental influences and oxidative hair treatments often lead to impaired combability of dry and wet hair. Furthermore, the gloss and moisture balance are disadvantageously affected by the attack on the external structure of the keratinic fibers. A further consequence of repeated treatments of keratinic fibers with surfactants and/or oxidative agents is marked grease reabsorption by the keratinic fibers as well as a strong tendency towards increased dandruff formation.

It is therefore an object of the present invention to reduce the side effects of environmental influences and of oxidative as well as surfactant hair treatments preferably during the oxidative or surfactant hair treatment itself but also after the oxidative or surfactant hair treatment, without impairing the effectiveness of the oxidative or surfactant cosmetic, in particular in terms of color intensity, colorfastness, lightening performance or waving action, as well as to prevent grease reabsorption by the keratinic fibers and increased dandruff formation. In addition, the oxidative treatment of keratin-containing fibers, in particular human hair, can also be combined with the application of effective protection of the fibers from environmental influences, e.g. UV protection, in one application step in the form of a 2-in-1 product. In particular, the object consisted in improving significantly the adhesion of fragrances to the keratinic fibers compared with compositions of the prior art. A further object consisted in maintaining the scent impression of the fragrance over a prolonged period from several hours up to at least 24 hours, even under negative environmental influences. Surprisingly, this is achieved with the combination of active agents according to the invention.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A hair treatment agent, containing, based on its weight, 0.01 to 15 wt. % of a fatty acid amide according to formula (I)

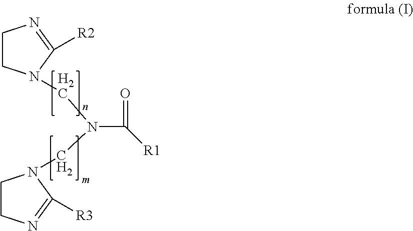

in which R1, R2 and R3 independently of one another denote a linear branched or unbranched C6 to C30, preferably C8 to C24, more preferably C12 to C22 and most preferably C12 to C18 alkyl or alkenyl group, wherein furthermore R2 preferably equals R3 and most preferably R1 equals R2 equals R3, and n and m independently of one another denote integers from 1 to 10, preferably 2 to 6 and most preferably 2, 3 and/or 4, wherein it is most preferred that n=m; and 0.001 to 5.0 wt. % of at least one fragrance.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

Fatty acid amides are chemical compounds that are known in principle and are likewise already used as ingredients in haircare agents. Fragrances per se and in compositions for application onto keratinic fibers are likewise known to the person skilled in the art. However, these known compositions cannot achieve the objects satisfactorily. Where the active agent complex is mentioned below, this refers to the ingredients a) and b) which must obligatorily be contained in the hair treatment agents according to the invention.

The active agent complex according to the invention leads to an improvement in brightness, an improvement in gloss, an improvement in the moisture balance as well as to protection from the destructuring of keratin-containing fibers, in particular human hair, by UV radiation and particularly to protection from oxidative damage, in particular to maintaining hair growth, preventing hair loss and preventing grease reabsorption by the keratinic fibers as well as to increasing the washfastness of colored keratinic fibers. In particular, the scent impression created by the fragrances is retained for a prolonged period, i.e. at least up to 24 hours.

The present invention therefore firstly provides a hair treatment agent containing—based on its weight—
a) 0.01 to 15 wt. %, based on the total composition, of at least one fatty acid amide according to formula (I)

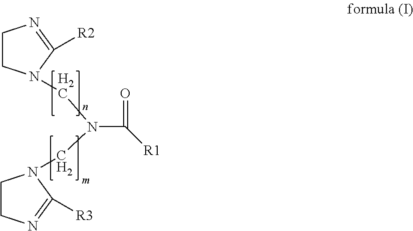

in which R1, R2 and R3 independently of one another denote a linear branched or unbranched C6 to C30, preferably C8 to C24, more preferably C12 to C22 and most preferably C12 to C18 alkyl or alkenyl group, wherein furthermore R2 preferably equals R3 and most preferably R1 equals R2 equals R3, and n and m independently of one another denote integers from 1 to 10, preferably 2 to 6 and most preferably 2, 3 and/or 4, wherein it is most preferred that n=m, and
b) 0.001 to 5.0 wt. % of at least one fragrance.

In a preferred embodiment of the present invention, the cosmetic agents are used to treat keratinic fibers, in particular human hair. Preferred agents according to the invention are therefore e.g. shampoos, hair coloring agents, conditioning agents or hair tonics.

Handle is defined as the tactile properties of a collective of fibers, with the person skilled in the art feeling and evaluating the parameters of fullness and suppleness of the collective by sensory means.

Shaping is understood to be the ability to impart a change in shape to a collective of previously treated keratin-containing fibers, in particular human hair. In hair cosmetics, the term “styling” is also used.

Maintenance of the natural growth of keratinic fibers is understood to mean that the influences on natural hair growth by hair cosmetic treatments as set out above, in particular by oxidative hair treatments, are compensated and there are no effects, or minor effects at most, on the natural growth of the keratinic fibers with respect to growth in thickness and length and/or with respect to the fullness of the hair.

An oxidative hair treatment is defined according to the invention as the action on hair of an oxidative cosmetic agent containing at least one oxidizing agent in a cosmetic carrier.

Retaining the scent of the treated keratinic fibers is understood to mean that the olfactory impression left behind by a fragrance on the keratinic fibers can be perceived olfactorily even during exposure to other scents, such as e.g. cold smoke, at least 24 hours after application of the cosmetic agent.

The hair treatment agent containing the active agent complex according to the invention is preferably used immediately before, during or after the oxidative or surfactant hair treatment. “Immediately before the oxidative or surfactant hair treatment” is understood within the meaning of the invention as an application which is followed directly by the oxidative or surfactant hair treatment, the hair treatment agent containing the active agent complex according to the invention having been previously rinsed from the hair or preferably left on the hair and the hair preferably being still wet.

“After the oxidative or surfactant hair treatment” is understood within the meaning of the invention as an application which either follows the oxidative or surfactant hair treatment directly, in which case the hair treatment agent containing the active agent complex according to the invention is applied onto the preferably still wet, towel-dry hair after rinsing off the agent having an oxidative or surfactant action, or is applied onto the dry or wet hair only after several hours or days. In both cases, the hair treatment agent according to the invention can be rinsed out again after a period of exposure from a few seconds up to 45 minutes or can remain on the hair completely.

The action of the hair treatment agent according to the invention even develops during the oxidative or surfactant hair treatment and surprisingly persists even after intensively washing out the hair treatment agent according to the invention.

The active agent complex according to the invention is preferably used in a cosmetic carrier. The cosmetic carriers can in particular be aqueous or aqueous-alcoholic. An aqueous cosmetic carrier contains at least 50 wt. % water. Aqueous-alcoholic cosmetic carriers within the meaning of the present invention are to be understood as aqueous solutions containing 3 to 70 wt. % of a C₁-C₆ alcohol, in particular methanol, ethanol or propanol, isopropanol, butanol, isobutanol, tert-butanol, n-pentanol, isopentanols, n-hexanol, isohexanols, glycol, glycerol, 1,2-pentanediol, 1,5-pentanediol, 1,2-hexanediol or 1,6-hexanediol. The agents according to the invention can additionally contain further organic solvents, such as e.g. methoxybutanol, benzyl alcohol, ethyl diglycol or 1,2-propylene glycol. All water-soluble organic solvents are preferred here.

The component a) of the active agent complex according to the invention is a fatty acid amide of the general formula (I)

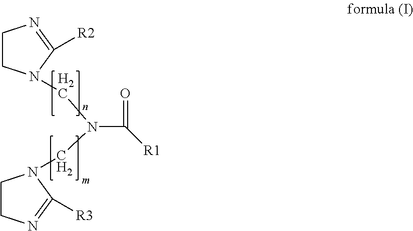

in which R1, R2 and R3 independently of one another denote a linear branched or unbranched C6 to C30, preferably C8 to C24, more preferably C12 to C22 and extremely preferably C12 to C18 alkyl or alkenyl group. R1 to R3 preferably denote capryl, caprylyl, octyl, nonyl, decanyl, lauryl, myristyl, cetyl, stearyl, isostearyl, oleyl, behenyl or arachidyl. Furthermore, it applies more preferably that R2 equals R3 and extremely preferably R1 equals R2 equals R3. The letters n and m independently of one another denote integers from 1 to 10, preferably 2 to 6 and extremely preferably 2, 3 and/or 4, with n=m being extremely preferred. Extremely preferably, R1 equals R2 equals R3 and they are selected from capryl, caprylyl, octyl, nonyl, decanyl, lauryl, myristyl, cetyl, stearyl, isostearyl, oleyl, behenyl or arachidyl and n=m=2. Most preferably, R1=R2=R3 and is selected from lauryl, myristyl, cetyl, stearyl, isostearyl, oleyl, behenyl or arachidyl, with cetyl, stearyl, isostearyl, oleyl or behenyl being more preferred among these, and n=m=2. The most preferred compound of formula (I) is the one with the INCI name bisethyl(isostearylimidazoline) isostearamide. The latter compound is commercially available from Croda with the trade name Keradyn® HH.

The hair treatment agents according to the invention contain the fatty acid amides according to the invention preferably in a quantity of 0.01 to 15.0 wt. %, more preferably 0.1 to 10.0 wt. %, particularly preferably 0.1 to 7.5 wt. %, extremely preferably 0.3 to 5.0 wt. %, based in each case on the weight of the ready-to-use hair treatment agent.

The ingredient b), a fragrance, is the second essential component of the present invention. The definition of a fragrance within the meaning of the present application coincides with the conventional definition from the art, as can be taken from the RÖMPP Chemie Lexikon as of December 2007. According to this, a fragrance is a chemical compound having an odor and/or taste that excites the receptors of the hair cells (adequate stimulus). The physical and chemical properties necessary for this are a low molar mass of at most 300 g/mol, a high vapor pressure, minimal water solubility and high lipid solubility, as well as weak polarity and the presence of at least one osmophoric group in the molecule. In order to distinguish volatile low-molecular-weight substances that are usually, and also within the meaning of the present application, regarded and used not as fragrances but principally as solvents, such as e.g. ethanol, propanol, isopropanol and acetone, from fragrances according to the invention, fragrances according to the invention have a molar mass of 74 to 300 g/mol, contain at least one osmophoric group in the molecule and have an odor and/or taste, i.e. they excite the receptors of the hair cells of the olfactory system. A fragrance within the meaning of the present invention can be a single substance but also an extract or essential oil or a resin or resinoid. According to this definition of “fragrances”, multiple fragrances can also be used within the meaning of the invention. The term “fragrances” within the meaning of the present invention is not intended to mean perfumes and/or perfume compositions or scent compositions with scent constituents put together from different sources. An example is provided with the intention of clearly illustrating the complex issue. Star anise oil is a fragrance within the meaning of the present invention although this oil consists of 15 individual constituents. Star anise oil is obtained by steam distillation. The term “fragrance” within the meaning of the present invention does not cover the so-called “notes”. By “notes”, the perfumer means blends of different individual discrete constituents to form a particular scent body, e.g. a rose body. Furthermore, the term “fragrances” within the meaning of this invention does not cover substances which on the one hand generate an odor but on the other hand are also usually used for other purposes in cosmetic compositions. Besides the already mentioned compounds ethanol, propanol, isopropanol and acetone, these are in particular phenoxyethanol and benzyl alcohol. Phenoxyethanol is usually employed because of its preservative action. Benzyl alcohol is usually used as a solvent.

As fragrances it is possible to use e.g. all of the fragrances listed in the book “Die kosmetischen Präparate”, 4^{th} edition, volume I, Die Parfümerie, Verlag für chemische Industrie, H. Ziolkowsky KG, Augsburg, G. A. Nowak, chapter I and chapter II.

A fragrance can itself already evoke an emotion. Fragrances of this type can be preferred within the meaning of the present invention. Those fragrances that evoke a mood experienced as pleasant and positive can be more preferred.

A fragrance according to the invention can be e.g. a synthetic product of the type of the esters, ethers, aldehydes, ketones, alcohols and hydrocarbons. Fragrance compounds of the type of the esters are e.g. benzyl acetate, phenoxyethyl isobutyrate, p-tert.-butylcyclohexyl acetate, linalyl acetate, dimethyl benzyl carbinyl acetate (DMBCA), phenyl ethyl acetate, benzyl acetate, ethyl methyl phenyl glycinate, allyl cyclohexyl propionate, styrallyl propionate, benzyl salicylate, cyclohexyl salicylate, Floramat, Melusat and Jasmacyclat. The ethers include e.g. benzyl ethyl ether and Ambroxan, the aldehydes include e.g. the linear alkanals with 8-18 C atoms, citral, citronellal, citronellyl oxyacetaldehyde, cyclamen aldehyde, lilial and bourgeonal, the ketones include e.g. the ionones, α-isomethyl ionone and methyl cedryl ketone, the alcohols include anethole, citronellol, eugenol, geraniol, linalool, phenyl ethyl alcohol and terpineol, and the hydrocarbons include mainly the terpenes, such as limonene and pinene.

However, the fragrances can also contain natural fragrances, as can be obtained from plant sources, e.g. pine, citrus, jasmine, patchouli, rose or ylang-ylang oils. Likewise suitable are clary sage oil, chamomile oil, clove oil, melissa oil, mint oil, cinnamon leaf oil, lime blossom oil, juniper berry oil, vetiver oil, olibanum oil, galbanum oil and labdanum oil as well as orange blossom oil, neroli oil, orange peel oil and sandalwood oil.

Tenacious fragrances that can be employed advantageously within the framework of the present invention are e.g. the essential oils, such as angelica root oil, anise oil, arnica blossom oil, basil oil, bay oil, champaca blossom oil, silver fir oil, silver fir cone oil, elemi oil, eucalyptus oil, fennel oil, fir needle oil, galbanum oil, geranium oil, gingergrass oil, guaiacwood oil, gurjun balsam oil, helichrysum oil, ho oil, ginger oil, iris oil, cajeput oil, calamus oil, chamomile oil, camphor oil, canaga oil, cardamom oil, cassia oil, pine needle oil, copaiva balsam oil, coriander oil, German spearmint oil, caraway oil, cumin oil, lemongrass oil, ambrette seed oil, myrrh oil, clove oil, neroli oil, niaouli oil, olibanum oil, oregano oil, palmarosa oil, patchouli oil, Peru balsam oil, petitgrain oil, pepper oil, peppermint oil, allspice oil, pine oil, rose oil, rosemary oil, sandalwood oil, celery oil, star anise oil, thuja oil, thyme oil, verbena oil, vetiver oil, juniper berry oil, wormwood oil, wintergreen oil, ylang-ylang oil, hyssop oil, cinnamon oil, cinnamon leaf oil and cypress oil.

However, the higher-boiling or solid fragrances of natural or synthetic origin can also be employed advantageously as tenacious fragrances within the framework of the present invention. These compounds include the following compounds as well as mixtures thereof: ambrettolide, α-amyl cinnamaldehyde, anethole, anisaldehyde, anisyl alcohol, anisole, anthranilic acid methyl ester, acetophenone, benzyl acetone, benzaldehyde, benzoic acid ethyl ester, benzophenone, benzyl alcohol, borneol, bornyl acetate, α-bromostyrene, n-decyl aldehyde, n-dodecyl aldehyde, eugenol, eugenol methyl ether, eucalyptol, farnesol, fenchone, fenchyl acetate, geranyl acetate, geranyl formate, heliotropin, heptyne carboxylic acid methyl ester, heptaldehyde, hydroquinone dimethyl ether, hydroxycinnamaldehyde, hydroxycinnamyl alcohol, indole, irone, isoeugenol, isoeugenol methyl ether, isosafrole, jasmone, camphor, carvacrol, carvone, p-cresol methyl ether, coumarin, p-methoxyacetophenone, methyl n-amyl ketone, methyl anthranilic acid methyl ester, p-methyl acetophenone, methyl chavicol, p-methyl quinoline, methyl β-naphthyl ketone, methyl n-nonyl acetaldehyde, methyl n-nonyl ketone, muscone, β-naphthol ethyl ether, β-naphthol methyl ether, nerol, nitrobenzene, n-nonyl aldehyde, nonyl alcohol, n-octyl aldehyde, p-oxyacetophenone, pentadecanolide, β-phenyl ethyl alcohol, phenyl acetaldehyde dimethyl acetal, phenylacetic acid, pulegone, safrole, salicylic acid isoamyl ester, salicylic acid methyl ester, salicylic acid hexyl ester, salicylic acid cyclohexyl ester, santalol, skatole, terpineol, thymene, thymol, γ-undecalactone, vanillin, veratrum aldehyde, cinnamaldehyde, cinnamyl alcohol, cinnamic acid, cinnamic acid ethyl ester, cinnamic acid benzyl ester.

The more volatile fragrances that can be employed advantageously within the framework of the present invention include in particular the lower-boiling fragrances of natural or synthetic origin, which can be employed singly or in mixtures. Examples of more volatile fragrances are alkyl isothiocyanates (alkyl mustard oils), butanedione, limonene, lilial, linalool, linalyl acetate and propionate, menthol, menthone, methyl n-heptenone, phellandrene, phenylacetaldehyde, terpinyl acetate, citral, citronellal. All of the aforementioned fragrances can be employed according to the present invention singly or in a mixture with the advantages already mentioned. In particular, fragrances from the group of the allyl alcohol esters, esters of secondary alcohols, esters of tertiary alcohols, allylic ketones, acetals, ketals, condensation products of amines and aldehydes and/or mixtures thereof can also be used.

Examples of particularly suitable fragrances within the meaning of the invention are bergamot oil, coriander oil, Damascenia, ginger oil, orange oil, patchouli oil, rosemary oil, peppermint oil, tamarin, geraniol, geranium oil, eugenol, mandarin and orange oil, anise, jasmine, isoeugenol, menthol, coumarin, melonal, terpineol, vanillin, ylang-ylang oil, cinnamyl alcohol, methyl ionone, ethyl vanillin, ethyl 2,6,6-trimethylcyclohexadiene carboxylate, methyl 2-nonenoate, butanedione, limonene, lilial, linalool, linalyl acetate and propionate, menthol, menthone, methyl n-heptenone, phellandrene, phenylacetaldehyde, terpinyl acetate, citral, citronellal and citronellol as well as mixtures thereof.

Most preferred is the use of citral, citronellal and citronellol, orange oil, patchouli oil, geraniol, geranium oil, eugenol, mandarin and orange oil, limonene, lilial, linalool and linalyl acetate as well as mixtures thereof.

At least one fragrance is contained in the cosmetic agent according to the invention in a total quantity of 0.001 to 5 wt. %, preferably 0.01 to 5 wt. %, more preferably 0.1 to 5 wt. %, extraordinarily preferably 0.3 to 5 wt. %, based in each case on the total weight of the agent.

A further most preferred ingredient in the hair treatment agents is a hydroxycarboxylic acid. Hydroxycarboxylic acids are carboxylic acids which have both at least one carboxy group and at least one hydroxy group in the molecule. In particular, the hydroxycarboxylic acids include the so-called AHA acids as well as β-hydroxycarboxylic acids. Another name for these hydroxy acids is fruit acid, because the acids are often found in fruit. The hydroxycarboxylic acid according to the invention is selected in particular from glycolic acid, lactic acid, glyceric acid, malic acid, citric acid, isocitric acid, mandelic acid, tartronic acid, tartaric acid, vanillic acid, salicylic acid, mevalonic acid, β-hydroxybutyric acid, gallic acid or protocatechuic acid. More preferably, the hydroxycarboxylic acid is selected from glycolic acid, lactic acid, glyceric acid, malic acid, citric acid, mandelic acid, tartaric acid, vanillic acid and salicylic acid. Particularly preferably, the hydroxycarboxylic acid is selected from glycolic acid, lactic acid, malic acid, citric acid, mandelic acid, tartaric acid and vanillic acid. Most preferably, the hydroxycarboxylic acid is selected from malic acid, mandelic acid, tartaric acid and vanillic acid. Mixtures of hydroxycarboxylic acids can, of course, also be used.

The compositions according to the invention contain the hydroxycarboxylic acids in a total quantity of 0.01 to 15.0 wt. %, in particular 0.01 to 10.0 wt. %, preferably 0.1 to 7.5 wt. % and most preferably in a quantity of 0.1 to 5.0 wt. %, based in each case on the total composition.

In a more preferred embodiment of the present invention, further selected quaternary ammonium compounds are preferably used with the compulsory ingredients already described above.

Quaternary ammonium compounds are in principle monomeric cationic or amphoteric ammonium compounds, monomeric amines, aminoamides, polymeric cationic ammonium compounds as well as polymeric amphoteric ammonium compounds. From this large number of possible quaternary ammonium compounds, the following groups have proved particularly suitable and each, taken by itself, is employed in a quantity of 0.1 to 15.0 wt. %. The quantity does not exceed or fall below these levels even if a mixture of different compounds of the quaternary ammonium compounds is used.

Cationic surfactants of the formula (Tkat1-1) make up the first group of cationic surfactants.

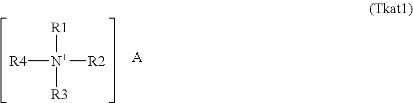

In the formula (Tkat1), R1, R2, R3 and R4 each independently of one another denote hydrogen, a methyl group, a phenyl group, a benzyl group or a saturated, branched or unbranched alkyl residue with a chain length of 8 to 30 carbon atoms, which may optionally be substituted with one or more hydroxy groups. A denotes a physiologically acceptable anion, e.g. halides such as chloride or bromide as well as methosulfates.

Examples of compounds of the formula (Tkat1) are lauryltrimethylammonium chloride, cetyltrimethylammonium chloride, cetyltrimethylammonium bromide, cetyltrimethylammonium methosulfate, dicetyldimethylammonium chloride, tricetylmethylammonium chloride, stearyltrimethylammonium chloride, distearyldimethylammonium chloride, lauryldimethylbenzylammonium chloride, behenyltrimethylammonium chloride, behenyltrimethylammonium bromide, behenyltrimethylammonium methosulfate.

Esterquats according to the formula (Tkat2) make up a preferred group.

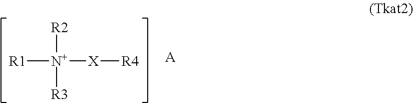

The residues R1, R2 and R3 are each independent of one another here and can be the same or different. The residues R1, R2 and R3 signify:
a branched or unbranched alkyl residue with 1 to 4 carbon atoms, which can contain at least one hydroxyl group, or
a saturated or unsaturated, branched or unbranched or a cyclic saturated or unsaturated alkyl residue with 6 to 30 carbon atoms, which can contain at least one hydroxyl group, or
an aryl or alkaryl residue, e.g. phenyl or benzyl,
the residue (—X—R4), with the proviso that no more than 2 of the residues R1, R2 or R3 can denote this residue.

The residue —(X—R4) is contained at least 1 to 3 times.

X here denotes:
1) —(CH2)n— with n=1 to 20, preferably n=1 to 10 and more preferably n=1-5, or
2) —(CH2—CHR5—O)n— with n=1 to 200, preferably 1 to 100, more preferably 1 to 50, and more preferably 1 to 20 with R5 in the meaning of hydrogen, methyl or ethyl,
3) a hydroxyalkyl group with one to four carbon atoms, which can be branched or unbranched and which contains at least one and no more than 3 hydroxy groups. Examples are: —CH₂OH, —CH₂CH₂OH, —CHOHCHOH, —CH₂CHOHCH₃, —CH(CH₂OH)₂, —COH(CH₂OH)₂, —CH₂CHOHCH₂OH, —CH₂CH₂CH₂OH and hydroxybutyl residues,
   and R4 denotes:
1) R6-O—CO, where R6 is a saturated or unsaturated, branched or unbranched or a cyclic saturated or unsaturated alkyl residue with 6 to 30 carbon atoms, which can contain at least one hydroxy group and which can optionally furthermore be ethoxylated with 1 to 100 ethylene oxide units and/or 1 to 100 propylene oxide units, or
2) R7-CO, where R7 is a saturated or unsaturated, branched or unbranched or a cyclic saturated or unsaturated alkyl residue with 6 to 30 carbon atoms, which can contain at least one hydroxy group and which can optionally furthermore be ethoxylated with 1 to 100 ethylene oxide units and/or 1 to 100 propylene oxide units,
   and A denotes a physiologically acceptable organic or inorganic anion and is defined at this point in representation of all the structures described, including those below. The anion in all of the cationic compounds described is selected from the halide ions, fluoride, chloride, bromide, iodide, sulfates of the general formula RSO₃—, where R has the meaning of saturated or unsaturated alkyl residues with 1 to 4 carbon atoms, or anionic residues of organic acids such as maleate, fumarate, oxalate, tartrate, citrate, lactate or acetate.

Products of this type are marketed e.g. with the trademarks Rewoquat®, Stepantex®, Dehyquart®, Armocare® and Akypoquat®. The products Armocare® VGH-70, Dehyquart® F-75, Dehyquart® C-4046, Dehyquart® L80, Dehyquart® F-30, Dehyquart® AU-35, Rewoquat® WE18, Rewoquat® WE38 DPG, Stepantex® VS 90 and Akypoquat® 131 are examples of these esterquats.

Other more preferred compounds according to the invention of the formula (Tkat1-2) are included in the formula (Tkat2.1), the cationic betaine esters.

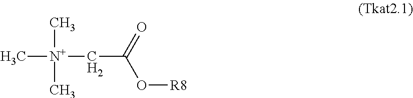

R8 corresponds in its meaning to R7.

More preferred are the esterquats with the trade names Armocare® VGH-70, as well as Dehyquart® F-75, Dehyquart® L80, Stepantex® VS 90 and Akypoquat® 131.

In preferred agents according to the invention, cationic surfactants of the formula (bI) are employed within relatively narrow quantitative ranges, so that preferred hair treatment agents according to the invention are characterized in that they contain 0.1 to 15 wt. %, preferably 0.5 to 10 wt. %, more preferably 1 to 10 wt. %, still more preferably 1.5 to 10 wt. % and in particular 2 to 5 wt. % of at least one compound of the general formula (I)

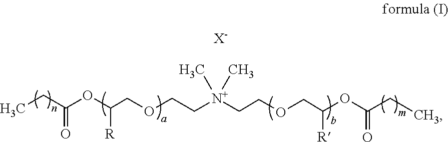

in which
n and m independently of one another denote integers between 5 and 40, with the proviso that n+m≧38; more preferably n=m; most preferably n=m=20;
a and b independently of one another denote integers between 1 and 10; independently of one another they particularly denote 1, 2, 3, 4 or 5; the equation a+2≧b≧a −2 preferably applies here and most preferably a=b=3;
R and R′ are selected independently of one another from —H and —CH₃; R═R′ preferably applies, so that preferably either PEG or PPG diesterquats are employed; R═R′=—CH₃ particularly preferably applies;
X⁻ is a physiologically acceptable anion, a halide such as chloride, bromide or iodide, toluenesulfonate, methosulfate etc., and is more preferably methosulfate.

In particular with the use of one of the compounds of formula (I) as described above, it has been shown that the care effects of the agents according to the invention can be further enhanced and in particular the stability of the agents can be further improved if, in addition to the compound(s) of formula (I), the agents contain certain acylated diamines.

Preferred hair treatment agents according to the invention are therefore characterized in that they additionally contain 0.1 to 10 wt. % of at least one compound of formula (II)

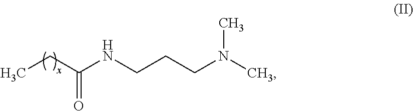

in which x denotes 18, 19, 20, 21, 22, 23 or 24.

Compounds of formula (II) with n=20 are more preferred here. Most preferred agents according to the invention are characterized in that they always contain a compound of formula (I) together with a compound of general formula (II).

Quaternary imidazoline compounds are another group. The formula (Tkat2) illustrated below shows the structure of these compounds.

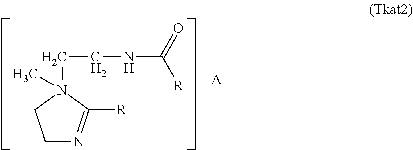

The residues R independently of one another each denote a saturated or unsaturated, linear or branched hydrocarbon residue with a chain length of 8 to 30 carbon atoms. The preferred compounds of the formula (Tkat2) each contain the same hydrocarbon residue for R. The chain length of the residues R is preferably 12 to 21 carbon atoms. A denotes an anion as described above. Examples that are particularly in accordance with the invention are available e.g. with the INCI names quaternium-27, quaternium-72, quaternium-83 and quaternium-91. Most preferred according to the invention is quaternium-91.

In a more preferred embodiment of the invention, the agents according to the invention furthermore contain at least one amine and/or cationized amine, in particular an amidoamine and/or a cationized amidoamine with the following structural formulae:

R1—NH—(CH₂)n—N⁺R²R³R⁴A (Tkat3)

where R1 signifies an acyl or alkyl residue with 6 to 30 C atoms, which can be branched or unbranched, saturated or unsaturated, and wherein the acyl residue and/or the alkyl residue can contain at least one OH group, and
R2, R3 and R4 each independently of one another signify
1) hydrogen or
2) an alkyl residue with 1 to 4 C atoms, which can be the same or different, saturated or unsaturated, and
3) a branched or unbranched hydroxyalkyl group with one to 4 carbon atoms with at least one and no more than three hydroxy groups, e.g. —CH₂OH, —CH₂CH₂OH, —CHOHCHOH, —CH₂CHOHCH3, CH(CH₂OH)₂, —COH(CH₂OH)₂, —CH₂CHOHCH₂OH, —CH₂CH₂CH₂OH and hydroxybutyl residues, and
   A signifies an anion as described above and
   n signifies an integer between 1 and 10.

Preferred is a composition in which the amine and/or the quaternized amine according to the general formula (Tkat3) is an amidoamine and/or a quaternized amidoamine, where R1 signifies a branched or unbranched, saturated or unsaturated acyl residue with 6 to 30 C atoms, which can contain at least one OH group. Preferred here is a fatty acid residue from oils and waxes, in particular from natural oils and waxes. As examples of these, lanolin, beeswax or candellila wax are suitable.

Preferred are also those amidoamines and/or quaternized amidoamines in which R2, R3 and/or R4 in the formula (Tkat3) signify a residue according to the general formula CH₂CH₂OR5, where R5 can have the meaning of alkyl residues with 1 to 4 carbon atoms, hydroxyethyl or hydrogen. The preferred value of n in the general formula (Tkat8) is an integer between 2 and 5.

The alkylamidoamines can either be present as such or can be converted to a quaternary compound in the composition by protonation in an appropriately acidic solution. Preferred according to the invention are the cationic alkylamidoamines.

Examples of commercial products of this type according to the invention are Witcamine® 100, Incromine® BB, Mackine® 401 and other Mackine® grades, Adogen® S18V, and as permanently cationic aminoamines: Rewoquat® RTM 50, Empigen® CSC, Swanol® Lanoquat DES-50, Rewoquat® UTM 50, Schercoquat® BAS, Lexquat® AMG-BEO or Incroquat® Behenyl HE.

The above-mentioned cationic surfactants can be used individually or in any combinations with one another, with quantities of between 0.01 to 10 wt. %, preferably in quantities of 0.01 to 7.5 wt. % and particularly preferably in quantities of 0.1 to 5.0 wt. % contained. The best results of all are obtained in this case with quantities of 0.1 to 3.0 wt. %, based in each case on the total composition of the respective agent.

Other quaternary ammonium compounds are cationic and amphoteric polymers.

The cationic and/or amphoteric polymers can be horn- or copolymers or polymers based on natural polymers, wherein the quaternary nitrogen groups are contained either in the polymer chain or preferably as a substituent on one or more of the monomers. The monomers containing ammonium groups can be copolymerized with non-cationic monomers. Suitable cationic monomers are unsaturated compounds capable of free-radical polymerization, which carry at least one cationic group, in particular ammonium-substituted vinyl monomers, such as e.g. trialkylmethacryloxyalkylammonium, trialkylacryloxyalkylammonium, dialkyldiallylammonium and quaternary vinylammonium monomers with cyclic groups containing cationic nitrogens, such as pyridinium, imidazolium or quaternary pyrrolidones, e.g. alkylvinylimidazolium, alkylvinylpyridinium or alkylvinylpyrrolidone salts. The alkyl groups of these monomers are preferably lower alkyl groups, such as e.g. C1 to C7 alkyl groups, more preferably C1 to C3 alkyl groups.

The monomers containing ammonium groups can be copolymerized with non-cationic monomers. Suitable comonomers are e.g. acrylamide, methacrylamide; alkyl and dialkyl acrylamide, alkyl and dialkyl methacrylamide, alkyl acrylate, alkyl methacrylate, vinyl caprolactone, vinylcaprolactam, vinylpyrrolidone, vinyl esters, e.g. vinyl acetate, vinyl alcohol, propylene glycol or ethylene glycol, the alkyl groups of these monomers preferably being C1 to C7 alkyl groups, particularly preferably C1 to C3 alkyl groups.

From the large number of these polymers, the following have proved to be particularly effective constituents of the active agent complex according to the invention: homopolymers of the general formula-{CH₂-[CR¹COO—(CH₂)ₘN⁺R²R³R⁴]}ₙX⁻,

in which R¹=—H or —CH₃, R², R³ and R⁴ independently of one another are selected from C1-4 alkyl, alkenyl or hydroxyalkyl groups, m=1, 2, 3 or 4, n is a natural number and X⁻ is a physiologically acceptable organic or inorganic anion. Among these polymers, those for which at least one of the following conditions applies are preferred according to the invention: R¹ denotes a methyl group, R², R³ and R⁴ denote methyl groups, m has the value 2.

As physiologically acceptable counterions X⁻, e.g. halide ions, sulfate ions, phosphate ions, methosulfate ions as well as organic ions such as lactate, citrate, tartrate and acetate ions are suitable. Preferred are methosulfates and halide ions, in particular chloride.

Suitable cationic polymers are e.g. copolymers according to the formula (Copo), which are contained in the hair treatment agents according to the invention preferably in a quantity—based on their weight—of 0.001 to 5 wt. %, for preference 0.0025 to 2.5 wt. %, more preferably 0.005 to 1 wt. %, particularly preferably 0.0075 to 0.75 wt. % and in particular 0.01 to 0.5 wt. %.

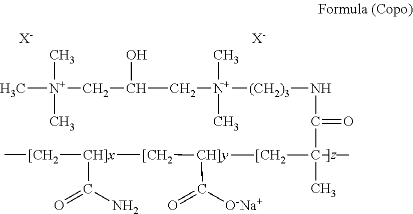

in which the following applies:
x+y+z=Q
Q denotes values from 3 to 55,000, preferably from 10 to 25,000, more preferably from 50 to 15,000, particularly preferably from 100 to 10,000, more particularly preferably from 500 to 8,000 and in particular from 1,000 to 5,000,
x denotes (0 to 0.5) Q, preferably (0 to 0.3) Q and in particular the values 0, 1, 2, 3, 4, 5, with the value 0 being preferred,
y denotes (0.1 to 0.95) Q, preferably (0.5 to 0.7) Q and in particular values of 1 to 24,000, preferably of 5 to 15,000, more preferably of 10 to 10,000 and in particular of 100 to 4,800,
z denotes (0.001 to 0.5) Q, preferably (0.1 to 0.5) Q and in particular values of 1 to 12,500, preferably of 2 to 8,000, more preferably of 3 to 4,000 and in particular of 5 to 2,000.

Regardless of which of the preferred copolymers of the formula (Copo) are employed, hair treatment agents according to the invention are preferred which are characterized in that the ratio of (y:z) is 4:1 to 1:2, preferably 4:1 to 1:1.

Regardless of which copolymers are employed in the agents according to the invention, hair treatment agents according to the invention are preferred in which the copolymer has a molar mass of 10,000 to 20 million gmol⁻¹, preferably of 100,000 to 10 million gmol⁻¹, more preferably of 500,000 to 5 million gmol⁻¹ and in particular of 1.1 million to 2.2 million gmol⁻¹.

A most preferred copolymer, which is constructed as set out above, is commercially available with the name polyquaternium-74.

A particularly suitable homopolymer is the optionally crosslinked poly(methacryloyloxyethyl trimethylammonium chloride) with the INCI name polyquaternium-37. Products of this type are commercially available e.g. with the names Rheocare® CTH (Cosmetic Rheologies) and Synthalen® CR (3V Sigma).

The homopolymer is preferably employed in the form of a non-aqueous polymer dispersion. Polymer dispersions of this type are commercially available with the names Salcare® SC 95 and Salcare® SC 96.

Suitable cationic polymers that are derived from natural polymers are cationic derivatives of polysaccharides, e.g. cationic derivatives of cellulose, starch or guar. Also suitable are chitosan and chitosan derivatives. Cationic polysaccharides have the general formula G-O—B—N+RₐR_{b}R_{c}A⁻

G is an anhydroglucose residue, e.g. starch or cellulose anhydroglucose;
B is a group of divalent compounds, e.g. alkylene, oxyalkylene, polyoxyalkylene or hydroxyalkylene;
Rₐ, R_{b} and R_{c} are, independently of one another, alkyl, aryl, alkylaryl, arylalkyl, alkoxyalkyl or alkoxyaryl, each with up to 18 C atoms, wherein the total number of the C atoms in Rₐ, R_{b} and R_{c} is preferably no more than 20;
A⁻ is a conventional counter-anion and is preferably chloride.

Cationic, i.e. quaternized, celluloses are available on the market with different degrees of substitution, cationic charge densities, nitrogen contents and molecular weights. For example, polyquaternium-67 is offered for sale with the names Polymer® SL or Polymer® SK (Amerchol). Another most preferred cellulose is offered with the trade name Mirustyle® CP from Croda. This is a trimonium and cocodimonium hydroxyethyl cellulose as a derivatized cellulose with the INCI name polyquaternium-72. Polyquaternium-72 can be used either in solid form or already pre-dissolved in aqueous solution.

Other cationic celluloses are Polymer JR® 400 (Amerchol, INCI name polyquaternium-10) as well as Polymer Quatrisoft® LM-200 (Amerchol, INCI name polyquatemium-24). Other commercial products are the compounds Celquat® H 100 and Celquat® L 200. More preferred cationic celluloses are polyquaternium-24, polyquaternium-67 and polyquaternium-72.

Suitable cationic guar derivatives are marketed with the trade name Jaguar and have the INCI name guar hydroxypropyltrimonium chloride. Particularly suitable cationic guar derivatives are also commercially available from Hercules with the name N-Hance®. Other cationic guar derivatives are marketed by Cognis with the name Cosmedia®. A preferred cationic guar derivative is the commercial product AquaCat® from Hercules. This raw material is a pre-dissolved cationic guar derivative. The cationic guar derivatives are preferred according to the invention.

A suitable chitosan is marketed e.g. by Kyowa Oil & Fat, Japan, with the trade name Flonac®. A preferred chitosan salt is chitosonium pyrrolidone carboxylate, which is marketed e.g. by Amerchol, USA, with the name Kytamer® PC. Other chitosan derivatives are readily available on the market with the trade names Hydagen® CMF, Hydagen® HCMF and Chitolam® NB/101.

Another group of polymers that are highly suitable for use according to the invention are polymers based on glucose. The following figure shows a cationic alkyl oligoglucoside of this type.

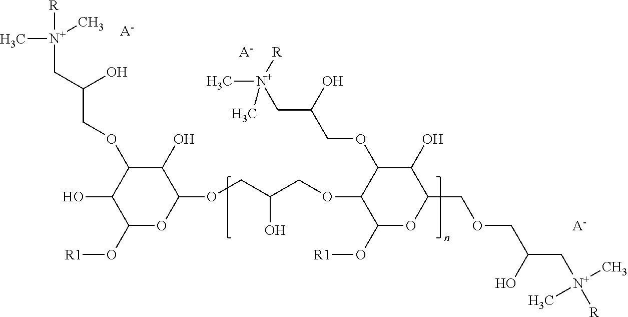

In the formula illustrated above, the residues R independently of one another denote a linear or branched C6 to C30 alkyl residue, a linear or branched C6-C30 alkenyl residue, and the residue R preferably denotes a residue R selected from: lauryl, myristyl, cetyl, stearyl, oleyl, behenyl or arachidyl.

The residues R1 independently of one another denote a linear or branched C6 to C30 alkyl residue, a linear or branched C6 to C30 alkenyl residue, and the residue R preferably denotes a residue selected from: butyl, capryl, caprylyl, octyl, nonyl, decanyl, lauryl, myristyl, cetyl, stearyl, oleyl, behenyl or arachidyl. More preferably, the residues R1 are the same. Still more preferably, the residues R1 are selected from technical mixtures of the fatty alcohol blends of C6/C8 fatty alcohols, C8/C10 fatty alcohols, C10/C12 fatty alcohols, C12/C14 fatty alcohols, C12/C18 fatty alcohols, and most preferred here are those technical fatty alcohol blends that are of vegetable origin. The counterion to the cationic charge is a physiologically acceptable anion, e.g. halide, methosulfate, phosphate, citrate, tartrate, etc. The counterion is preferably a halide, such as fluoride, chloride, bromide or methosulfate. Most preferably, the anion is chloride.

More preferred examples of the cationic alkyl oligoglucosides are the compounds with the INCI names polyquaternium-77, polyquaternium-78, polyquaternium-79, polyquaternium-80, polyquatemium-81 and polyquaternium-82. Most preferred are the cationic alkyl oligoglucosides with the names polyquaternium-77, polyquatemium-81 and polyquaternium-82.

Compounds of this type can be purchased e.g. with the name Poly Suga® Quat from Colonial Chemical Inc.

The cationic alkyl oligoglucosides are used in a total quantity of 0.01 to 10.0 wt. %, preferably of 0.05 to 5.0 wt. %, still more preferably of 0.1 to 3.0 wt. % and most preferably in quantities of 0.2 to 2.0 wt. %, based in each case on the total weight of the composition. It is also included in the invention, of course, that more mixtures of cationic alkyl oligoglucosides can be used. In this case, it is preferred if in each case a long-chain and a short-chain cationic alkyl oligoglucoside are used simultaneously.

Another preferred cationic polymer can be obtained on the basis of ethanolamine. The polymer is commercially available with the name polyquatemium-71.

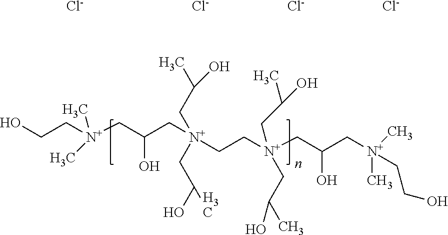

This polymer can be purchased e.g. with the name Cola® Moist 300 P from Colonial Chemical Inc.

Polyquaternium-71 is used in a total quantity of 0.01 to 10.0 wt. %, preferably of 0.05 to 5.0 wt. %, still more preferably of 0.1 to 3.0 wt. % and most preferably in quantities of 0.2 to 2.0 wt. %, based in each case on the total weight of the composition.

Furthermore, with particular preference, a cationic alkyl oligoglucoside, as shown in the following figure, can be used.

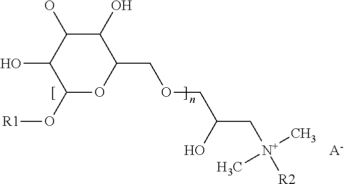

In the formula illustrated above, the residue R2 denotes a linear or branched C6 to C30 alkyl residue, a linear or branched C6-C30 alkenyl residue, and preferably the residue R denotes a residue R selected from: lauryl, myristyl, cetyl, stearyl, oleyl, behenyl or arachidyl.

The residue R1 denotes a linear or branched C6 to C30 alkyl residue, a linear or branched C6 to C30 alkenyl residue and preferably the residue R1 denotes a residue selected from: butyl, capryl, caprylyl, octyl, nonyl, decanyl, lauryl, myristyl, cetyl, stearyl, oleyl, behenyl or arachidyl. Still more preferably, the residue R1 is selected from technical mixtures of the fatty alcohol blends of C6/C8 fatty alcohols, C8/C10 fatty alcohols, C10/C12 fatty alcohols, C12/C14 fatty alcohols, C12/C18 fatty alcohols, and most preferred here are those technical fatty alcohol blends which are of vegetable origin. The index n denotes a number between 1 and 20, preferably between 1 and 10, more preferably between 1 and 5 and most preferably between 1 and 3. The counterion to the cationic charge, A⁻, is a physiologically acceptable anion, e.g. halide, methosulfate, phosphate, citrate, tartrate, etc. The counterion is preferably a halide, such as fluoride, chloride, bromide or methosulfate. Most preferably, the anion is chloride.

More preferred examples of the cationic alkyl oligoglucosides are the compounds with the INCI names laurdimoniumhydroxypropyl decylglucosides chloride, laurdimoniumhydroxypropyl laurylglucosides chloride, stearyldimoniumhydroxypropyl decylglucosides chloride, stearyldimoniumhydroxypropyl laurylglucosides chloride, stearyldimoniumhydroxypropyl laurylglucosides chloride or cocoglucosides hydroxypropyltrimonium chloride.

Compounds of this type can be purchased e.g. with the name Suga® Quat from Colonial Chemical Inc.

The cationic alkyl oligoglucosides are used in a total quantity of 0.01 to 10.0 wt. %, preferably of 0.05 to 5.0 wt. %, still more preferably of 0.1 to 3.0 wt. % and most preferably in quantities of 0.2 to 2.0 wt. %, based in each case on the total weight of the composition. It is also included in the invention, of course, that more mixtures of cationic alkyl oligoglucosides can be used. In this case, it is preferred if in each case a long-chain and a short-chain cationic alkyl oligoglucoside are used simultaneously.

A more preferred cationic polymer according to the invention is the copolymer of N-vinylpyrrolidone, N-vinylcaprolactam, N-(3-dimethylaminopropyl)methacrylamide and 3-(methacryloylamino)propyl lauryl dimethylammonium chloride (INCI name: polyquaternium-69), which is marketed e.g. by ISP with the trade name AquaStyle® 300 (28-32 wt. % active substance in an ethanol-water mixture, molecular weight 350,000).

Other preferred cationic polymers are e.g.
cationized honey, e.g. the commercial product Honeyquat® 50,
polymeric dimethyldiallylammonium salts and copolymers thereof with esters and amides of acrylic acid and methacrylic acid. The products that are commercially available with the names Merquat® 100 (poly(dimethyldiallylammonium chloride)) and Merquat® 550 (dimethyldiallylammonium chloride-acrylamide copolymer) are examples of these cationic polymers with the INCI name polyquaternium-7,
vinylpyrrolidone-vinylimidazolium methochloride copolymers, as offered with the names Luviquat® FC 370, FC 550 and the INCI name polyquatemium-16 as well as FC 905 and HM 552,
quaternized vinylpyrrolidone/dimethylaminoethyl methacrylate, e.g. vinylpyrrolidone/dimethylaminoethyl methacrylate methosulfate copolymer, which is marketed with the trade names Gafquat® 755 N and Gafquat® 734 by Gaf Co., USA, and the INCI name polyquatemium-11,
quaternized polyvinyl alcohol,
as well as the polymers known by the names polyquaternium-2, polyquaternium-17, polyquatemium-18 and polyquaternium-27 with quaternary nitrogen atoms in the polymer main chain,
vinylpyrrolidone-vinylcaprolactam-acrylate terpolymers, as offered for sale with acrylic acid esters and acrylic acid amides as the third monomer building block, e.g. with the name Aquaflex® SF 40.

Amphoteric polymers according to the invention are those polymers in which a cationic group is derived from at least one of the following monomers:
(i) monomers with quaternary ammonium groups of the general formula (Mono1), R¹—CH═CR²—CO—Z—(CₙH₂ₙ)—N⁽⁺⁾R²R³R⁴ (Mono1)
   in which R¹ and R² independently of one another denote hydrogen or a methyl group and R³, R⁴ and R⁵ independently of one another denote alkyl groups with 1 to 4 carbon atoms, Z is an NH group or an oxygen atom, n is an integer from 2 to 5 and A⁽⁻⁾ is the anion of an organic or inorganic acid,
(ii) monomers with quaternary ammonium groups of the general formula (Mono2),

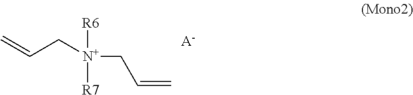

where R⁶ and R⁷ independently of one another denote a (C₁ to C₄) alkyl group, in particular a methyl group, and
A⁻ is the anion of an organic or inorganic acid,
(iii) monomeric carboxylic acids of the general formula (Mono3),
   R⁸—CH═CR⁹—COOH (Mono3)
   in which R⁸ and R⁹ independently of one another are hydrogen or methyl groups.

More preferred are those polymers in which monomers of type (i) are employed in which R³, R⁴ and R⁵ are methyl groups, Z is an NH group and A⁽⁻⁾ is a halide, methoxysulfate or ethoxysulfate ion; acrylamidopropyltrimethylammonium chloride is a more preferred monomer (i). As monomer (ii) for the above-mentioned polymers, acrylic acid is preferably used.

More preferred amphoteric polymers are copolymers of at least one monomer (Mono1) or (Mono2) with the monomer (Mono3), in particular copolymers of the monomers (Mono2) and (Mono3). Particularly preferably used amphoteric polymers according to the invention are copolymers of diallyldimethylammonium chloride and acrylic acid. These copolymers are marketed with the INCI name polyquatemium-22, inter alia with the trade name Merquat® 280 (Nalco).

In addition, besides a monomer (Mono1) or (Mono2) and a monomer (Mono3), the amphoteric polymers according to the invention can additionally contain a monomer (Mono4)

(iv) monomeric carboxylic acid amides of the general formula (Mono4),

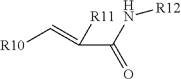

in which R¹⁰ and R¹¹ independently of one another are hydrogen or methyl groups and R¹² denotes a hydrogen atom or a (C₁ to C₈) alkyl group.

Particularly preferably used amphoteric polymers according to the invention based on a comonomer (Mono4) are terpolymers of diallyldimethylammonium chloride, acrylamide and acrylic acid. These copolymers are marketed with the INCI name polyquatemium-39, inter alia with the trade name Merquat® Plus 3330 (Nalco).

The amphoteric polymers can generally be employed according to the invention either directly or in the form of a salt, which is obtained by neutralization of the polymers, e.g. with an alkali hydroxide.

The above-mentioned cationic polymers can be used individually or in any combinations with one another, with quantities of between 0.01 to 10 wt. %, preferably quantities of 0.01 to 7.5 wt. % and particularly preferably in quantities of 0.1 to 5.0 wt. % being contained. The best results of all here are obtained with quantities of 0.1 to 3.0 wt. %, based in each case on the total composition of the respective agent.

The hair treatment agents according to the invention naturally also contain, besides the active agent combination according to the invention, other constituents that are conventional in cosmetic compositions. The selection of these constituents generally depends on the intended use of the hair treatment agents. In the case of a shampoo, for example, further surface-active substances will be contained. In the case of deep conditioners, further cationic compounds and further care substances will optionally be contained.

In a more preferred embodiment of the active agent complex according to the invention, the agents according to the invention preferably contain at least one amino functional silicone. These silicones can be described e.g. by the formula (Si-2)

M(RₐQ_{b}SiO_{(4-a-b/2})ₓ(R_{c}SiO_{(4-c)2})_{y}M (Si-2)

wherein in the above formula
R is a hydrocarbon or a hydrocarbon residue with 1 to about 6 carbon atoms,
Q is a polar residue of the general formula —R¹HZ, where
   R¹ is a divalent linking group, which is bound to hydrogen and the residue Z, made up of carbon and hydrogen atoms, carbon, hydrogen and oxygen atoms or carbon, hydrogen and nitrogen atoms, and
   Z is an organic, amino functional residue, which contains at least one amino functional group;
a assumes values in the range of about 0 to about 2,
b assumes values in the range of about 1 to about 3,
a+b is less than or equal to 3, and
c is a number in the range of about 1 to about 3, and
x is a number in the range of 1 to about 2,000, preferably of about 3 to about 50 and most preferably of about 3 to about 25, and
y is a number in the range of about 20 to about 10,000, preferably of about 125 to about 10,000 and most preferably of about 150 to about 1,000, and
M is a suitable silicone terminal group, as known in the prior art, preferably trimethylsiloxy.

Z according to formula (Si-2) is an organic, amino functional residue containing at least one functional amino group. A possible formula for said Z is NH(CH₂)_{z}NH₂, where z is an integer greater than or equal to 1. Another possible formula for said Z is —NH(CH₂)_{z}(CH₂)_{zz}NH, where both z and zz independently of one another are an integer greater than or equal to 1, this structure including diamino ring structures, such as piperazinyl. Said Z is most preferably an —NHCH₂CH₂NH₂ residue. Another possible formula for said Z is —N(CH₂)_{z}(CH₂)_{zz}NX₂ or —NX₂, where each X of X₂ is selected independently from the group consisting of hydrogen and alkyl groups with 1 to 12 carbon atoms, and zz is 0.

Q according to formula (Si-2) is most preferably a polar amino functional residue of the formula —CH₂CH₂CH₂NHCH₂CH₂NH₂.

In the formula (Si-2), a assumes values in the range of 0 to 2, b assumes values in the range of 2 to 3, a+b is less than or equal to 3, and c is a number in the range of 1 to 3. Suitable according to the invention are cationic silicone oils, such as e.g. the commercially available products Dow Corning (DC) 929 Emulsion, DC 2-2078, DC 5-7113, SM-2059 (General Electric) as well as SLM-55067 (Wacker).

More preferred agents according to the invention are characterized in that they contain at least one amino functional silicone of the formula (Si3-a)

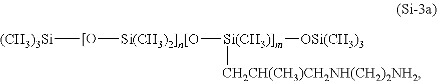

where m and n are numbers, the sum (m+n) of which is between 1 and 2000, preferably between 50 and 150, wherein n preferably assumes values of 0 to 1999 and in particular of 49 to 149 and m preferably assumes values of 1 to 2000, in particular of 1 to 10.

These silicones are designated according to the INCI Declaration as trimethylsilylamodimethicones and are available e.g. with the name Q2-7224 (manufacturer: Dow Corning; a stabilized trimethylsilylamodimethicone).

More preferred are also agents according to the invention which contain at least one amino functional silicone of the formula (Si-3b)

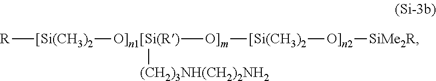

where
R denotes —OH, an (optionally ethoxylated and/or propoxylated) (C₁ to C₂₀) alkoxy group or a —CH₃ group,
R′ denotes —OH, a (C₁ to C₂₀) alkoxy group or a —CH3 group and
m, n1 and n2 are numbers, the sum (m+n1+n2) of which is between 1 and 2000, preferably between 50 and 150, wherein the sum (n1+n2) preferably assumes values of 0 to 1999 and in particular of 49 to 149 and m preferably assumes values of 1 to 2000, in particular of 1 to 10.

These silicones are designated according to the INCI Declaration as amodimethicones, or as functionalized amodimethicones, such as e.g. bis(C13-15 alkoxy) PG amodimethicones (available e.g. as the commercial product: DC 8500 from Dow Corning).

Suitable diquaternary silicones are selected from compounds of the general formula (Si3c)

[R¹R²R³N⁺-A-SiR⁷R⁸—(O—SiR⁹R¹⁰)ₙ—O—SiR¹¹R¹²-A-N⁺R⁴R⁵R⁶]2X⁻ (Si3c)

wherein the residues R1 to R6 independently of one another signify C1 to C22 alkyl residues, which can contain hydroxy groups, and wherein preferably at least one of the residues has at least 8 C atoms and the remaining residues have 1 to 4 C atoms, the residues R7 to R12 independently of one another are the same or different and signify C1 to C10 alkyl or phenyl, A signifies a divalent organic linking group,
n is a number from 0 to 200, preferably from 10 to 120, more preferably from 10 to 40, and X⁻ is an anion.

The divalent linking group is preferably a C1 to C12 alkylene or alkoxyalkylene group, which can be substituted with one or more hydroxyl groups. More preferred is the group —(CH₂)₃—O—CH₂—CH(OH)—CH₂—.

The anion X⁻ can be a halide ion, an acetate, an organic carboxylate or a compound of the general formula RSO₃—, where R has the meaning of C1 to C4 alkyl residues.

A preferred diquatemary silicone has the general formula (Si3d) [RN⁺Me₂-A-(SiMe₂O)ₙ— SiMe₂-A-N⁺Me₂R]2 CH₃COO⁻(Si3d),

wherein A is the group —(CH₂)₃—O—CH₂— CH(OH)—CH₂,
R is an alkyl residue with at least 8 C atoms and n is a number from 10 to 120.

Suitable silicone polymers with two terminal, quaternary ammonium groups are known, with the INCI name quaternium-80. These are dimethylsiloxanes with two terminal trialkylammonium groups. Diquaternary polydimethylsiloxanes of this type are marketed by Evonik with the trade names Abil® Quat 3270, 3272 and 3474.

Preferred hair treatment agents according to the invention are characterized in that they contain, based on their weight, 0.01 to 10 wt. %, preferably 0.01 to 8 wt. %, more preferably 0.1 to 7.5 wt. % and in particular 0.2 to 5 wt. % amino functional silicone(s) and/or diquatemary silicone.

Polyammonium-polysiloxane compounds are another silicone according to the invention having amino functions. The polyammonium-polysiloxane compounds can be purchased e.g. from GE Bayer Silicones with the trade name Baysilone®. The products with the names Baysilone TP 3911, SME 253 and SFE 839 are preferred here. Particularly preferred is the use of Baysilone TP 3911 as an active component of the compositions according to the invention. The polyammonium-polysiloxane compounds are used in the compositions according to the invention in a quantity of 0.01 to 10 wt. %, preferably 0.01 to 7.5, more preferably 0.01 to 5.0 wt. %, particularly preferably of 0.05 to 2.5 wt. %, based on the total composition in each case.

EP 1887024 A1 describes novel cationic amino functional silicones, which in particular improve the gloss in agents for caring for surfaces, e.g. human hair. These cationic silicone polymers are distinguished by the fact that they have a silicone framework as well as at least one polyether part and also at least one part with an ammonium structure. Examples of the preferred cationic silicone polymers within the meaning of the present invention, besides the compounds of the above-mentioned EP 1887024 A1, are furthermore in particular the compounds with the INCI names: silicone quaternium-1, silicone quaternium-2, silicone quaternium-3, silicone quaternium-4, silicone quaternium-5, silicone quaternium-6, silicone quaternium-7, silicone quaternium-8, silicone quaternium-9, silicone quaternium-10, silicone quaternium-11, silicone quaternium-12, silicone quaternium-15, silicone quaternium-16, silicone quaternium-17, silicone quaternium-18, silicone quaternium-20, silicone quaternium-21, silicone quaternium-22 as well as silicone quaternium-2 panthenol succinate and silicone quaternium-16/glycidyl dimethicone crosspolymer. Most preferred is in particular silicone quaternium-22. This raw material is marketed e.g. by Evonik with the trade name Abil® T-Quat 60.

A final more preferred amino silicone according to the invention corresponds to the following formula:

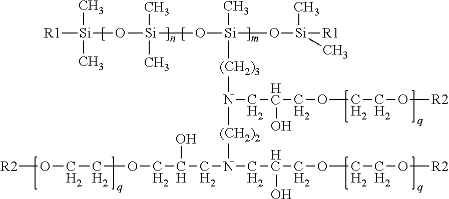

in which R1 denotes a methyl, ethyl, hydroxy, methoxy or ethoxy group,
R2 denotes a straight-chained or branched C8 to C24 alkyl or alkylene residue, preferably a straight-chained or branched C9 to C22 alkyl or alkenyl residue, more preferably a straight-chained or branched C11 to C18 alkyl or alkenyl residue, most preferably a corresponding alkyl residue,
n and m each denote integers from 1 to 1000 and
each q denotes an integer from 2 to 50, preferably 4 to 30, more preferably 4 to 18 and most preferably from 4 to 12.

The molecular weight of these compounds is 15,000 to 2,000,000, measured with a Brookfield rotational viscometer RV, spindle 5, at 20° C. The molecular weight is preferably 30,000 to 1,750,000 and more preferably 50,000 to 1,500,000. The nitrogen content of the silicones according to the invention is 0.03 to 4.2 wt. %, preferably 0.1 to 2.8 wt. % and most preferably 0.16 to 1.4 wt % Amino functional cationic silicones according to the invention of the above formula can be purchased e.g. from Clariant. A most preferred product according to the invention is commercially available with the INCI name “trideceth-9-amodimethicone and trideceth-12”.

The cationic amino functional silicone polymers of the formula described above are contained in the compositions according to the invention in quantities of 0.01 to 5 wt. %, preferably in quantities of 0.05 to 5 wt. % and particularly preferably in quantities of 0.1 to 5 wt. %. The best results of all are obtained here with quantities of 0.1 to 2.5 wt. %, based in each case on the total composition of the respective agent.

The cationic amino functional silicone polymers are contained in the compositions according to the invention in quantities of 0.01 to 10 wt. %, preferably in quantities of 0.05 to 10 wt. % and particularly preferably in quantities of 0.1 to 7.5 wt. %. The best results of all are obtained here with quantities of 0.1 to 5 wt. %, based in each case on the total composition of the respective agent.

The dimethicones according to the invention can be either linear or branched or cyclic or cyclic and branched. Linear dimethicones can be illustrated by the following structural formula (Si1):

(SiR¹₃)—O—(SiR²₂—O—)ₓ(Si¹₃) (Si1)

Branched dimethicones can be illustrated by the structural formula (Si1.1):

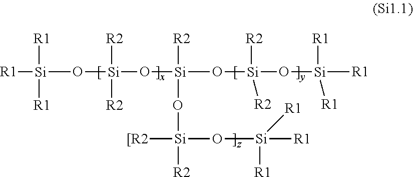

The residues R¹ and R² independently of one another each denote hydrogen, a methyl residue, a C2 to C30 linear, saturated or unsaturated hydrocarbon residue, a phenyl residue and/or an aryl residue. The numbers x, y and z are integers and each independently of one another run from 0 to 50,000. The molecular weights of the dimethicones are between 1000 D and 10,000,000 D. The viscosities are between 100 and 10,000,000 cPs measured at 25° C. using a glass capillary viscometer by the Dow Corning Corporate Test Method CTM 0004 of 20 Jul. 1970. Preferred viscosities are between 1000 and 5,000,000 cPs; particularly preferred viscosities are between 10,000 and 3,000,000 cPs. The most preferred range is between 50,000 and 2,000,000 cPs. Extremely preferred are viscosities around the range of approximately 60,000 cPs. The product “Dow Corning 200 with 60,000 cSt” may be referred to here by way of example.

More preferred cosmetic or dermatological preparations according to the invention are characterized in that they contain at least one silicone of the formula (Si1.2)

(CH₃)₃Si—[O—Si(CH₃)₂]ₓ—O—Si(CH₃)₃ (Si1.2),

in which x denotes a number from 0 to 100, preferably from 0 to 50, more preferably from 0 to 20 and in particular 0 to 10.

The dimethicones (Si1) are contained in the compositions according to the invention in quantities of 0.01 to 10 wt. %, preferably 0.01 to 8 wt. %, particularly preferably 0.1 to 7.5 wt. % and in particular 0.1 to 5 wt. %, based on the total composition.

Finally, the silicone compounds are understood to include the dimethiconols (Si8). The dimethiconols can be either linear or branched or cyclic or cyclic and branched. Linear dimethiconols can be illustrated by the following structural formula (Si8-I):

(SiOHR¹₂)—O—(SiR²₂—O —)ₓ—(SiOHR¹₂) (Si8-I)

Branched dimethiconols can be illustrated by the structural formula (Si8-II):

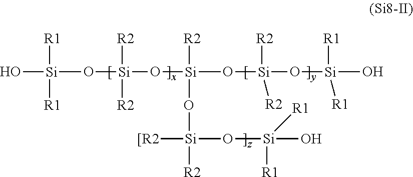

The residues R¹ and R² independently of one another each denote hydrogen, a methyl residue, a C2 to C30 linear, saturated or unsaturated hydrocarbon residue, a phenyl residue and/or an aryl residue. The numbers x, y and z are integers and each independently of one another run from 0 to 50,000. The molecular weights of the dimethiconols are between 1000 D and 10,000,000 D. The viscosities are between 100 and 10,000,000 cPs measured at 25° C. using a glass capillary viscometer by the Dow Corning Corporate Test Method CTM 0004 of 20 Jul. 1970. Preferred viscosities are between 1000 and 5,000,000 cPs; particularly preferred viscosities are between 10,000 and 3,000,000 cPs. The most preferred range is between 50,000 and 2,000,000 cPs.

As examples of these products, the following commercial products are mentioned: Dow Corning 1-1254 Fluid, Dow Corning 2-9023 Fluid, Dow Corning 2-9026 Fluid, Abil OSW 5 (Degussa Care Specialties), Dow Corning 1401 Fluid, Dow Corning 1403 Fluid, Dow Corning 1501 Fluid, Dow Corning 1784 HVF Emulsion, Dow Corning 9546 Silicone Elastomer Blend, SM555, SM2725, SM2765, SM2785 (all four of the above-mentioned: GE Silicones), Wacker-Belsil CM 1000, Wacker-Belsil CM 3092, Wacker-Belsil CM 5040, Wacker-Belsil DM 3096, Wacker-Belsil DM 3112 VP, Wacker-Belsil DM 8005 VP, Wacker-Belsil DM 60081 VP (all of the above-mentioned: Wacker-Chemie GmbH).

The dimethiconols (Si8) are in the compositions according to the invention in quantities of 0.01 to 10 wt. %, preferably 0.01 to 8 wt. %, particularly preferably 0.1 to 7.5 wt. % and in particular 0.1 to 5 wt. % dimethiconol, based on the composition.

The cyclic dimethicones referred to according to INCI as cyclomethicones can also be employed with preference according to the invention. In this case, cosmetic or dermatological preparations according to the invention are preferred which contain at least one silicone of the formula (Si-4)

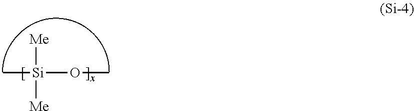

in which x denotes a number from 3 to 200, preferably from 3 to 10, more preferably from 3 to 7 and in particular 3, 4, 5 or 6.

Agents that are likewise preferred according to the invention are characterized in that they contain at least one silicone of the formula (Si-5)

R₃Si-[O—SiR₂]ₓ—(CH₂)ₙ-[O—SiR₂]_{y}—O—SiR₃ (Si-5),

in which R denotes identical or different residues from the group —H, -phenyl, -benzyl, —CH₂—CH(CH₃)Ph, the C₁₋₂₀ alkyl residues, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, x and y respectively denote a number from 0 to 200, preferably from 0 to 10, more preferably from 0 to 7 and in particular 0, 1, 2, 3, 4, 5 or 6, and n denotes a number from 0 to 10, preferably from 1 to 8 and in particular 2, 3, 4, 5, 6.

As further silicones besides the dimethicones, dimethiconols, amodimethicones and/or cyclomethicones according to the invention, water-soluble silicones can be contained in the compositions according to the invention.

Appropriate hydrophilic silicones are selected e.g. from the compounds of the formulae (Si-6) and/or (Si-7). Particularly preferred water-soluble, silicone-based surfactants are selected from the group of the dimethicone copolyols, which are preferably alkoxylated, in particular polyethoxylated or polypropoxylated.

Dimethicone copolyols are understood according to the invention preferably as polyoxyalkylene-modified dimethylpolysiloxanes of the general formulae (Si-6) or (Si-7):

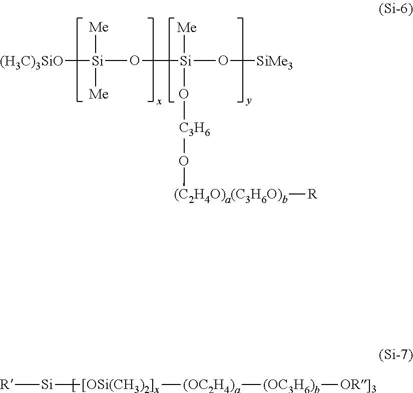

where the residue R denotes a hydrogen atom, an alkyl group with 1 to 12 C atoms, an alkoxy group with 1 to 12 C atoms or a hydroxyl group, the residues R′ and R″ signify alkyl groups with 1 to 12 C atoms, x denotes an integer from 1 to 100, preferably from 20 to 30, y denotes an integer from 1 to 20, preferably from 2 to 10 and a and b denote integers from 0 to 50, preferably from 10 to 30.

More preferred dimethicone copolyols within the meaning of the invention are e.g. the products marketed commercially with the trade name SILWET (Union Carbide Corporation) and DOW CORNING. More preferred dimethicone copolyols according to the invention are Dow Corning 190 and Dow Corning 193.

The dimethicone copolyols are in the compositions according to the invention in quantities of 0.01 to 10 wt. %, preferably 0.01 to 8 wt. %, more preferably 0.1 to 7.5 wt. % and in particular 0.1 to 5 wt. % of dimethicone copolyol, based on the composition.

Although all silicones can be used with the active agent combination according to the invention, it has been shown that the effect declines in the sequence: amino functional silicones; dimethicones comparable with dimethiconols; cyclomethicones and water-soluble silicones. With the use of more than two silicones, combinations of amino functional silicones with dimethicones and/or dimethiconols have been shown to be most effective in increasing the effect of the active agent combination according to the invention. The best effects of all are obtained if the more preferred silicone compounds already described in each case are used as silicones.

Another ingredient that increases the effect of the active agent combination according to the invention is an oil substance. With particular preference, these are e.g. ester oils. The ester oils are defined as follows:

Ester oils are to be understood as the esters of C₆-C₃₀ fatty acids with C₂-C₃₀ fatty alcohols. Preferred are the monoesters of the fatty acids with alcohols having 2 to 24 C atoms. Examples of fatty acid portions employed in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid as well as technical mixtures thereof. Examples of the fatty alcohol portions in the ester oils are isopropyl alcohol, caproyl alcohol, capryl alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, linoleyl alcohol, linolenyl alcohol, elaeostearyl alcohol, arachyl alcohol, gadoleyl alcohol, behenyl alcohol, erucyl alcohol and brassidyl alcohol as well as technical mixtures thereof. More preferred according to the invention are isopropyl myristate (Rilanit® IPM), isononanoic acid C16-18 alkyl ester (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN) and oleic acid decyl ester (Cetiol® V).

The ester oils can, of course, also be alkoxylated with ethylene oxide, propylene oxide or mixtures of ethylene oxide and propylene oxide. The alkoxylation can be found in this case either on the fatty alcohol part or on the fatty acid part or on both parts of the ester oils. However, it is preferred according to the invention if the fatty alcohol was first alkoxylated and then esterified with fatty acid. Formula (D4-II) gives a general illustration of these compounds.

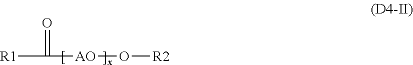

R1 here denotes a saturated or unsaturated, branched or unbranched, cyclic saturated cyclic unsaturated acyl residue with 6 to 30 carbon atoms,

AO denotes ethylene oxide, propylene oxide or butylene oxide,
X denotes a number between 1 and 200, preferably 1 and 100, more preferably between 1 and 50, particularly preferably between 1 and 20, extremely preferably between 1 and 10 and most preferably between 1 and 5,
R2 denotes a saturated or unsaturated, branched or unbranched, cyclic saturated cyclic unsaturated alkyl, alkenyl, alkynyl, phenyl or benzyl residue with 6 to 30 carbon atoms. Examples of fatty acid portions employed as residue R1 in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid as well as technical mixtures thereof. Examples of the fatty alcohol portions as residue R2 in the ester oils are benzyl alcohol, isopropyl alcohol, caproyl alcohol, capryl alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, linoleyl alcohol, linolenyl alcohol, elaeostearyl alcohol, arachyl alcohol, gadoleyl alcohol, behenyl alcohol, erucyl alcohol and brassidyl alcohol as well as technical mixtures thereof. A more preferred ester oil according to the invention is available e.g. with the INCI name PPG-3 benzyl ether myristate.

The following are also to be understood as ester oils:
dicarboxylic acid esters, such as di-n-butyl adipate, di(2-ethylhexyl) adipate, di(2-ethylhexyl) succinate and diisotridecyl acetate, as well as diol esters, such as ethylene glycol dioleate, ethylene glycol diisotridecanoate, propylene glycol di(2-ethylhexanoate), propylene glycol diisostearate, propylene glycol dipelargonate, butanediol diisostearate, neopentyl glycol dicaprylate, as well as
symmetrical, asymmetrical or cyclic esters of carbonic acid with fatty alcohols, e.g. glycerol carbonate or dicaprylyl carbonate (Cetiol® CC),
trifatty acid esters of saturated and/or unsaturated linear and/or branched fatty acids with glycerol,
fatty acid partial glycerides, i.e. monoglycerides, diglycerides and technical mixtures thereof. Typical examples are mono- and/or diglycerides based on caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid as well as technical mixtures thereof. Oleic acid monoglycerides are preferably employed.

The ester oils are used in the agents according to the invention in a quantity of 0.01 to 20 wt. %, preferably 0.01 to 10.0 wt. %, more preferably 0.01 to 7.5 wt. %, most preferably of 0.1 to 5.0 wt. %. It is, of course, also possible according to the invention to use multiple ester oils at the same time.

Other oil substances according to the invention are:
vegetable oils. Examples of these oils are sunflower oil, olive oil, soybean oil, rapeseed oil, almond oil, jojoba oil, orange oil, wheat germ oil, peach kernel oil and the liquid portions of coconut oil. However, other triglyceride oils, such as the liquid portions of beef tallow, as well as synthetic triglyceride oils are also suitable;
liquid paraffin oils, isoparaffin oils and synthetic hydrocarbons as well as di-n-alkyl ethers with a total of between 12 and 36 C atoms, in particular 12 to 24 C atoms, such as e.g. di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether and n-hexyl-n-undecyl ether as well as di-tert-butyl ether, diisopentyl ether, di-3-ethyldecyl ether, tert.-butyl-n-octyl ether, isopentyl-n-octyl ether and 2-methylpentyl-n-octyl ether. The compounds 1,3-di-(2-ethylhexyl) cyclohexane (Cetiol® S) and di-n-octyl ether (Cetiol® OE), which are available as commercial products, may be preferred.

As natural oils, e.g. amaranth seed oil, apricot kernel oil, argan oil, avocado oil, babassu oil, cottonseed oil, borage seed oil, camelina oil, safflower oil, peanut oil, pomegranate seed oil, grapefruit seed oil, hemp oil, hazelnut oil, elderberry seed oil, blackcurrant seed oil, jojoba oil, cocoa butter, linseed oil, macadamia nut oil, maize germ oil, almond oil, marula oil, evening primrose oil, olive oil, palm oil, rapeseed oil, rice oil, sea buckthorn berry oil, sea buckthorn seed oil, sesame oil, shea butter, soybean oil, sunflower oil, grape seed oil, walnut oil or wild rose oil are used.

In many cases, the agents contain at least one surface-active substance, with both anionic and zwitterionic, ampholytic, nonionic and cationic surface-active substances being suitable in principle. The selection of the surface-active substances depends on the nature of the agent.

As anionic surfactants (Tanion) in preparations according to the invention, all anionic surface-active substances suitable for use on the human body are suitable. Typical examples of anionic surfactants are:
linear and branched fatty acids with 8 to 30 C atoms (soaps),
ether carboxylic acids of the formula R—O—(CH₂—CH₂O)ₓ—CH₂—COOH, in which R is a linear alkyl group with 8 to 30 C atoms and x=0 or 1 to 16 and salts thereof,
acyl sarcosides with 8 to 24 C atoms in the acyl group,
acyl taurides with 8 to 24 C atoms in the acyl group,
acyl isethionates with 8 to 24 C atoms in the acyl group,
sulfosuccinic acid mono- and dialkyl esters with 8 to 24 C atoms in the alkyl group and sulfosuccinic acid monoalkyl polyoxyethyl esters with 8 to 24 C atoms in the alkyl group and 1 to 6 oxyethyl groups,
linear alkanesulfonates with 8 to 24 C atoms,
linear alpha-olefin sulfonates with 8 to 24 C atoms,
alpha-sulfo fatty acid methyl esters of fatty acids with 8 to 30 C atoms,
alkyl sulfates and alkyl polyglycol ether sulfates of the formula R—O(CH₂—CH₂O)ₓ—OSO₃H, in which R is a preferably linear alkyl group with 8 to 30 C atoms and x=0 or 1 to 12,
hydroxysulfonates substantially corresponding to at least one of the following two formulae or mixtures thereof as well as salts thereof, CH₃—(CH2)_{y}—CHOH—(CH₂)ₚ—(CH— SO₃M)-(CH₂), —CH₂—O—(CₙH₂ₙ)ₓ—H and/or CH₃—(CH₂)_{y}—(CH—SO₃M)-(CH₂)ₚ—CHOH—(CH₂), —CH₂—O—(CₙH₂ₙO)ₓ—H wherein in both formulae y and z=0 or integers from 1 to 18, p=0, 1 or 2 and the sum of (y+z+p) is a number from 12 to 18, x=0 or a number from 1 to 30 and n is an integer from 2 to 4, and M=H or alkali metal, in particular sodium, potassium, lithium, alkaline earth metal, in particular magnesium, calcium, zinc and/or an ammonium ion, which may optionally be substituted, in particular mono-, di-, tri- or tetraammonium ions with C1 to C4 alkyl, alkenyl or aryl residues,
sulfated hydroxyalkyl polyethylene and/or hydroxyalkylene propylene glycol ethers of the formula R¹—(CHOSO₃M)-CHR³—(OCHR⁴—CH₂)ₙ—OR² with R¹ denoting a linear alkyl residue with 1 to 24 C atoms, R² a linear or branched, saturated alkyl residue with 1 to 24 C atoms, R³ hydrogen or a linear alkyl residue with 1 to 24 C atoms, R⁴ hydrogen or a methyl residue and M denoting hydrogen, ammonium, alkylammonium, alkanolammonium, where the alkyl and alkanol residues each have 1 to 4 C atoms, or a metal atom selected from lithium, sodium, potassium, calcium or magnesium and n a number in the range of 0 to 12 and furthermore the total number of the C atoms contained in R¹ and R³ is 2 to 44,
sulfonates of unsaturated fatty acids with 8 to 24 C atoms and 1 to 6 double bonds,
esters of tartaric acid and citric acid with alcohols, which represent addition products of about 2-15 molecules ethylene oxide and/or propylene oxide to fatty alcohols with 8 to 22 C atoms,
alkyl and/or alkenyl ether phosphates of the formula R¹(OCH₂CH₂), —O—(PO—OX)—OR²,
in which R¹ preferably denotes an aliphatic hydrocarbon residue with 8 to 30 carbon atoms, R² denotes hydrogen, a (CH₂CH₂O)ₙR² residue or X, n denotes numbers from 1 to 10 and X denotes hydrogen, an alkali metal or alkaline earth metal or NR³R⁴R⁵R⁶, with R³ to R⁶ independently of one another denoting hydrogen or a C₁ to C₄ hydrocarbon residue,
sulfated fatty acid alkylene glycol esters of the formula RCO(AlkO)ₙSO₃M in which RCO— denotes a linear or branched, aliphatic, saturated and/or unsaturated acyl residue with 6 to 22 C atoms, Alk denotes CH₂CH₂, CHCH₃CH₂ and/or CH₂CHCH₃, n denotes numbers from 0.5 to 5 and M denotes a metal, such as alkali metal, in particular sodium, potassium, lithium, alkaline earth metal, in particular magnesium, calcium, zinc, or ammonium ion, such as ⁺NR³R⁴R⁵R⁶, with R³ to R⁶ independently of one another denoting hydrogen or a C₁ to C₄ hydrocarbon residue,
monoglyceride sulfates and monoglyceride ether sulfates of the formula R⁸OC—(OCH₂CH₂)ₓ—OCH₂-[CHO(CH₂CH₂O)_{y}H]-CH₂O(CH₂CH₂O)_{z}—SO₃X,
in which R⁸CO denotes a linear or branched acyl residue with 6 to 22 carbon atoms, x, y and z in total denote 0 or numbers from 1 to 30, preferably 2 to 10, and X denotes an alkali metal or alkaline earth metal. Typical examples of monoglyceride (ether) sulfates that are suitable within the meaning of the invention are the reaction products of lauric acid monoglyceride, coconut fatty acid monoglyceride, palmitic acid monoglyceride, stearic acid monoglyceride, oleic acid monoglyceride and tallow fatty acid monoglyceride as well as ethylene oxide adducts thereof with sulfur trioxide or chlorosulfonic acid in the form of their sodium salts. Monoglyceride sulfates are preferably employed in which R⁸CO denotes a linear acyl residue with 8 to 18 carbon atoms,
amide ether carboxylic acids, R¹—CO—NR²—CH₂CH₂—O—(CH₂CH₂O)ₙCH₂COOM, with R¹ as a straight-chained or branched alkyl or alkenyl residue with a number of carbon atoms in the chain of 2 to 30, n denotes an integer from 1 to 20 and R² denotes hydrogen, a methyl, ethyl, propyl, isopropyl, n-butyl, t-butyl or isobutyl residue and M denotes hydrogen or a metal such as alkali metal, in particular sodium, potassium, lithium, alkaline earth metal, in particular magnesium, calcium, zinc, or an ammonium ion, such as ⁺NR³R⁴R⁵R⁶, with R³ to R⁶ independently of one another denoting hydrogen or a C₁ to C₄ hydrocarbon residue. Products of this type are available e.g. from Chem-Y with the product name Akypo®;
acyl glutamates of the formula XOOC—CH₂CH₂CH(C(NH)OR)—COOX, in which RCO denotes a linear or branched acyl residue with 6 to 22 carbon atoms and 0 and/or 1, 2 or 3 double bonds and X denotes hydrogen, an alkali metal and/or alkaline earth metal, ammonium, alkylammonium, alkanolammonium or glucammonium,
condensation products of a water-soluble salt of a water-soluble protein hydrolyzate with a C8-C30 fatty acid. Products of this type have long been commercially available with the trade mark Lamepon®, Maypon®, Gluadin®, Hostapon® KCG or Amisoft®;
alkyl and/or alkenyl oligoglycoside carboxylates, sulfates, phosphates and/or isethionates,
acyl lactylates and
hydroxy mixed ether sulfates.

Where the mild anionic surfactants contain polyglycol ether chains, it is particularly preferred that these have a narrow homolog distribution. Furthermore, in the case of mild anionic surfactants with polyglycol ether units, it is preferred that the number of glycol ether groups is 1 to 20, preferably 2 to 15, more preferably 2 to 12. Particularly mild anionic surfactants with polyglycol ether groups without a restricted homolog distribution can also be contained if, for example, on the one hand, the number of polyglycol ether groups is 4 to 12 and Zn or Mg ions are selected as counterions. One example thereof is the commercial product Texapon® ASV.

Particularly suitable zwitterionic surfactants are the so-called betaines, such as the N-alkyl-N,N-dimethylammonium glycinates, e.g. cocoalkyl dimethylammonium glycinate, N-acylaminopropyl-N,N-dimethylammonium glycinates, e.g. cocoacyl aminopropyl dimethylammonium glycinate, and 2-alkyl-3-carboxymethyl-3-hydroxyethyl imidazolines each with 8 to 18 C atoms in the alkyl or acyl group as well as cocoacyl aminoethyl hydroxyethyl carboxymethyl glycinate. A preferred zwitterionic surfactant is the fatty acid amide derivative known by the INCI name cocamidopropyl betaine.

Ampholytic surfactants (Tampho) are understood to be those surface-active compounds that are capable of forming inner salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids each with about 8 to 24 C atoms in the alkyl group. Typical examples of amphoteric or zwitterionic surfactants are alkyl betaines, alkylamidobetaines, aminopropionates, aminoglycinates, imidazolinium betaines and sulfobetaines.

More preferred ampholytic surfactants are N-cocoalkyl aminopropionate, cocoacylaminoethyl aminopropionate and C₁₂-C₁₈ acylsarcosine.

Nonionic surfactants (Tnio) are e.g. addition products of 2 to 50 mol ethylene oxide and/or 0 to 5 mol propylene oxide to linear and branched fatty alcohols with 6 to 30 C atoms, the fatty alcohol polyglycol ethers or the fatty alcohol polypropylene glycol ethers or mixed fatty alcohol polyethers,
addition products of 2 to 50 mol ethylene oxide and/or 0 to 5 mol propylene oxide to linear and branched fatty acids with 6 to 30 C atoms, the fatty acid polyglycol ethers or the fatty acid polypropylene glycol ethers or mixed fatty acid polyethers,
addition products of 2 to 50 mol ethylene oxide and/or 0 to 5 mol propylene oxide to linear and branched alkylphenols with 8 to 15 C atoms in the alkyl group, the alkylphenol polyglycol ethers or the alkyl polypropylene glycol ethers, or mixed alkylphenol polyethers,
addition products of 2 to 50 mol ethylene oxide and/or 0 to 5 mol propylene oxide to linear and branched fatty alcohols with 8 to 30 C atoms, to fatty acids with 8 to 30 C atoms and to alkylphenols with 8 to 15 C atoms in the alkyl group, end-capped with a methyl or C₂-C₆ alkyl residue, such as e.g. the grades available with the trade names Dehydol® LS, Dehydol® LT (Cognis),
C₁₂-C₃₀ fatty acid mono- and diesters of addition products of 1 to 30 mol ethylene oxide to glycerol,
addition products of 5 to 60 mol ethylene oxide to castor oil and hydrogenated castor oil,
polyol fatty acid esters, such as e.g. the commercial product Hydagen® HSP (Cognis) or Sovermol® grades (Cognis),
alkoxylated triglycerides,
alkoxylated fatty acid alkyl esters of the formula (Tnio-1) R¹CO—(OCH₂CHR²)_{w}OR³ (Tnio-1)
in which R¹CO denotes a linear or branched, saturated and/or unsaturated acyl residue with 6 to 22 carbon atoms, R² denotes hydrogen or methyl, R³ denotes linear or branched alkyl residues with 1 to 4 carbon atoms and w denotes numbers from 1 to 20,
amine oxides,
hydroxy mixed ethers, R¹O[CH₂CH(CH₃)O]ₓ(CH₂CHR²O)_{y}[CH₂CH(OH)R³]_{z} with R¹ denoting a linear or branched, saturated or unsaturated alkyl and/or alkenyl residue with 2 to 30 C atoms, R² denoting hydrogen, a methyl, ethyl, propyl or isopropyl residue, R³ denoting a linear or branched alkyl residue with 2 to 30 C atoms, x denoting 0 or a number from 1 to 20, Y denoting a number from 1 to 30 and z denoting the number 1, 2, 3, 4 or 5,
sorbitan fatty acid esters and addition products of ethylene oxide to sorbitan fatty acid esters, such as e.g. the polysorbates,
sugar fatty acid esters and addition products of ethylene oxide to sugar fatty acid esters,
addition products of ethylene oxide to fatty acid alkanolamides and fatty amines,
sugar surfactants of the type of the alkyl and alkenyl oligoglycosides,
sugar surfactants of the type of the fatty acid N-alkyl polyhydroxyalkylamides,
fatty acid amide polyglycol ethers, fatty amine polyglycol ethers,
mixed ethers or mixed formals and polysorbates.

The surfactants (T) are employed in quantities of 0.05-45 wt. %, preferably 0.1-30 wt. % and particularly preferably of 0.5-25 wt. %, based on the total agent used according to the invention.

Emulsifiers that can be used according to the invention are e.g.
addition products of 4 to 30 mol ethylene oxide and/or 0 to 5 mol propylene oxide to linear fatty alcohols with 8 to 22 C atoms, to fatty acids with 12 to 22 C atoms and to alkylphenols with 8 to 15 C atoms in the alkyl group,
C₁₂-C₂₂ fatty acid mono- and diesters of addition products of 1 to 30 mol ethylene oxide to polyols with 3 to 6 carbon atoms, in particular to glycerol,
ethylene oxide and polyglycerol addition products to methyl glucoside fatty acid esters, fatty acid alkanolamides and fatty acid glucamides,
C₈-C₂₂ alkyl mono- and oligoglycosides and ethoxylated analogs thereof, wherein degrees of oligomerization of 1.1 to 5, in particular 1.2 to 2.0, and glucose as sugar component are preferred,
mixtures of alkyl (oligo)glucosides and fatty alcohols e.g. the commercially available product Montanov® 68,
addition products of 5 to 60 mol ethylene oxide to castor oil and hydrogenated castor oil,
partial esters of polyols having 3-6 carbon atoms with saturated fatty acids having 8 to 22 C atoms,
sterols, both from animal tissue (zoosterols, cholesterol, lanosterol) and from vegetable fats (phytosterols, ergosterol, stigmasterol, sitosterol) or from fungi and yeasts (mycosterols),
phospholipids (lecithins, phosphatidylcholines),
fatty acid esters of sugars and sugar alcohols, such as sorbitol,
polyglycerols and polyglycerol derivatives, such as e.g. polyglycerol poly-12-hydroxystearate (commercial product Dehymuls® PGPH).

The agents according to the invention contain the emulsifiers preferably in quantities of 0.1-25 wt. %, in particular 0.5-15 wt. %, based on the total agent.

With particular preference, the compositions according to the invention contain fats (Fat) as an additional active agent. Fats (Fat) are to be understood as fatty acids, fatty alcohols, natural and synthetic waxes, which can occur in both solid form and as a liquid in aqueous dispersion, and natural and synthetic cosmetic oil components.

As fatty acids (Fatac) it is possible to employ linear and/or branched, saturated and/or unsaturated fatty acids with 6-30 carbon atoms. Fatty acids with 10-22 carbon atoms are preferred. Among these, e.g. the isostearic acids, such as the commercial products Emersol® 871 and Emersol® 875, and isopalmitic acids, such as the commercial product Edenor® IP 95, should be mentioned, as well as all other fatty acids marketed with the trade names Edenor® (Cognis). Further typical examples of these fatty acids are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid as well as technical mixtures thereof. More preferred are usually the fatty acid blends, which are obtainable from coconut oil or palm oil; the use of stearic acid is generally particularly preferred.

The quantity employed in this case is 0.1-15 wt. %, based on the total agent. The quantity is preferably 0.5-10 wt. %, with quantities of 1-5 wt. % being particularly advantageous.

As fatty alcohols (Fatal) it is possible to employ saturated, mono- or polyunsaturated, branched or unbranched fatty alcohols with C₆-C₃₀, preferably C₁₀-C₂₂ and particularly preferably C₁₂-C₂₂ carbon atoms. Within the meaning of the invention it is possible to employ e.g. decanol, octanol, octenol, dodecenol, decenol, octadienol, dodecadienol, decadienol, oleyl alcohol, erucyl alcohol, ricinoleyl alcohol, stearyl alcohol, isostearyl alcohol, cetyl alcohol, lauryl alcohol, myristyl alcohol, arachidyl alcohol, capryl alcohol, capric alcohol, linoleyl alcohol, linolenyl alcohol and behenyl alcohol, as well as Guerbet alcohols thereof, this list being intended to be of an exemplary and non-limiting nature. However, the fatty alcohols originate from preferably natural fatty acids, in which case it can usually be assumed that they are obtained from the esters of the fatty acids by reduction. According to the invention it is also possible to employ those fatty alcohol blends that represent a mixture of different fatty alcohols. Substances of this type can be purchased e.g. with the names Stenol®, e.g. Stenol® 1618, or Lanette®, e.g. Lanette® O, or Lorol®, e.g. Lorol® C8, Lorol® C14, Lorol® C18, Lorol® C8-18, HD-Ocenol®, Crodacol®, e.g. Crodacol® CS, Novol®, Eutanol® G, Guerbitol® 16, Guerbitol® 18, Guerbitol® 20, Isofol® 12, Isofol® 16, Isofol® 24, Isofol® 36, Isocarb® 12, Isocarb® 16 or Isocarb® 24. It is, of course, also possible according to the invention to employ wool wax alcohols, as can be purchased e.g. with the names Corona®, White Swan®, Coronet® or Fluilan®. The fatty alcohols are employed in quantities of 0.1-30 wt. %, based on the total preparation, preferably in quantities of 0.1-20 wt. %.

As natural or synthetic waxes (Fatwax), it is possible according to the invention to employ solid paraffins or isoparaffins, carnauba waxes, beeswaxes, candelilla waxes, ozokerites, ceresin, spermaceti, sunflower wax, fruit waxes, such as e.g. apple wax or citrus wax, micro waxes of PE or PP. Waxes of this type are available e.g. through Kahl & Co., Trittau.

The quantity employed is 0.1-50 wt. %, based on the total agent, preferably 0.1-20 wt. % and more preferably 0.1-15 wt. %, based on the total agent.

The total quantity of oil and fat components in the agents according to the invention is usually 0.5-75 wt. %, based on the total agent. Quantities of 0.5-35 wt. % are preferred according to the invention.

Protein hydrolyzates and/or derivatives thereof are another synergistic active agent according to the invention in the compositions according to the invention with the active agent complex according to the invention.

According to the invention, protein hydrolyzates of both vegetable and animal or marine or synthetic origin can be employed.

Animal protein hydrolyzates are e.g. elastin, collagen, keratin, silk and milk protein hydrolyzates, which can also occur in the form of salts. Products of this type are marketed e.g. with the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda).

Furthermore, vegetable protein hydrolyzates such as e.g. soy, almond, pea, moringa, potato and wheat protein hydrolyzates are preferred according to the invention. Products of this type are available e.g. with the trademarks Gluadin® (Cognis), DiaMin® (Diamalt), Lexein® (Inolex), Hydrosoy® (Croda), Hydrolupin® (Croda), Hydrosesame® (Croda), Hydrotritium® (Croda), Crotein® (Croda) and Puricare® LS 9658 from Laboratoires Sérobiologiques.

Other preferred protein hydrolyzates according to the invention are of marine origin. These include e.g. collagen hydrolyzates from fish or algae as well as protein hydrolyzates from bivalves or pearl hydrolyzates. Examples of pearl extracts according to the invention are the commercial products Pearl Protein Extract BG® or Crodarom® Pearl.

Furthermore, cationized protein hydrolyzates may be included among the protein hydrolyzates and derivatives thereof, with the basic protein hydrolyzate originating from an animal, e.g. from collagen, milk or keratin, from a plant, e.g. from wheat, maize, rice, potatoes, soy or almonds, from marine life forms, e.g. from fish collagen or algae, or protein hydrolyzates obtained by biotechnology. As typical examples of the cationic protein hydrolyzates and derivatives according to the invention, the products that are mentioned with the INCI names in the “International Cosmetic Ingredient Dictionary and Handbook”, (seventh edition 1997, The Cosmetic, Toiletry and Fragrance Association, 1101 17^{th} Street, N.W., Suite 300, Washington, D.C. 200364702) and that are commercially available may be mentioned.

The protein hydrolyzates (P) are contained in the compositions in concentrations of 0.001 wt. % to 20 wt. %, preferably of 0.05 wt. % to 15 wt. % and particularly preferably in quantities of 0.05 wt. % to 5 wt. %.

Another preferred group of ingredients of the compositions according to the invention with the active agent complex according to the invention are vitamins, provitamins or vitamin precursors.

Vitamins, pro-vitamins and vitamin precursors that are allocated to the groups A, B, C, E, F and H are more preferred here.

The group of substances referred to as vitamin A includes retinol (vitamin A₁) and 3,4-didehydroretinol (vitamin A₂). β-Carotene is the provitamin of retinol. Suitable according to the invention as vitamin A component are e.g. vitamin A acid and esters thereof, vitamin A aldehyde and vitamin A alcohol and esters thereof, such as the palmitate and acetate. The agents according to the invention contain the vitamin A component preferably in quantities of 0.05-1 wt. %, based on the total preparation.

The vitamin B group or vitamin B complex includes, inter alia: Vitamin B₁ (thiamine); Vitamin B₂ (riboflavin); Vitamin B₃. This name often covers the compounds nicotinic acid and nicotinamide (niacinamide). Preferred according to the invention is nicotinamide, which is contained in the agents used according to the invention preferably in quantities of 0.05 to 1 wt. %, based on the total agent.

Vitamin B₅ (pantothenic acid, panthenol and pantolactone). Within the context of this group, preferably panthenol and/or pantolactone is/are used. Derivatives of panthenol that can be used according to the invention are in particular the esters and ethers of panthenol and cationically derivatized panthenols. Individual representatives are e.g. panthenol triacetate, panthenol monoethyl ether and monoacetate thereof as well as cationic panthenol derivatives. Pantothenic acid is preferably employed in the present invention as a derivative in the form of the more stable calcium salts and sodium salts (Ca pantothenate, Na pantothenate).

Vitamin B₆ (pyridoxine as well as pyridoxamine and pyridoxal).

The above compounds of the vitamin B type, in particular vitamin B₃, B₅ and B₆, are contained in the agents according to the invention preferably in quantities of 0.05-10 wt. %, based on the total agent. Quantities of 0.1-5 wt. % are more preferred.

Vitamin C (ascorbic acid). Vitamin C is employed in the agents according to the invention preferably in quantities of 0.1 to 3 wt. %, based on the total agent. Use in the form of the palmitic acid ester, glucosides or phosphates can be preferred. Use in combination with tocopherols can likewise be preferred.

Vitamin E (tocopherols, in particular α-tocopherol). Tocopherol and derivatives thereof; including in particular the esters, such as the acetate, the nicotinate, the phosphate and the succinate, are contained in the agents according to the invention preferably in quantities of 0.05-1 wt. %, based on the total agent.

Vitamin F. The term “vitamin F” is generally understood to mean essential fatty acids, in particular linoleic acid, linolenic acid and arachidonic acid.

Vitamin H. The compound (3aS,4S,6aR)-2-oxohexahydrothieno[3,4-d]-imidazole-4-valeric acid is referred to as vitamin H, but its trivial name biotin has now become accepted. Biotin is contained in the agents according to the invention preferably in quantities of 0.0001 to 1.0 wt. %, in particular in quantities of 0.001 to 0.01 wt. %.

The compositions according to the invention preferably contain vitamins, provitamins and vitamin precursors from the groups A, B, E and H. Panthenol, pantolactone, pyridoxine and derivatives thereof as well as nicotinamide and biotin are more preferred.

A more preferred group of ingredients in the cosmetic compositions according to the invention are the betaines mentioned below: carnitine, carnitine tartrate, carnitine magnesium citrate, acetyl carnitine, betalains, 1,1-dimethylproline, choline, choline chloride, choline bitartrate, choline dihydrogen citrate and the compound N,N,N-trimethylglycine, which is referred to in the literature as betaine.

Carnitine, histidine, choline and betaine are preferably used. In a more preferred embodiment of the invention, L-carnitine tartrate is employed as an active agent.

More preferred are agents according to the invention which contain—based on their weight—0.0001 to 10.0 wt. %, preferably 0.0005 to 5.0 wt. %, more preferably 0.001 to 2.0 wt. % and in particular 0.001 to 1.0 wt. % of at least one of the above-mentioned betaines, in particular carnitine tartrate.

In another preferred embodiment according to the invention, the compositions according to the invention contain bioquinones. In the agents according to the invention, suitable bioquinones are understood to be one or more ubiquinone(s) and/or plastoquinone(s). The preferred ubiquinones according to the invention have the following formula:

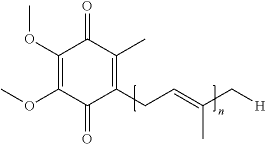

with n=6, 7, 8, 9 or 10.

The coenzyme Q-10 is most preferred here.

Preferred compositions according to the invention contain purine and/or purine derivatives in relatively narrow quantitative ranges. Preferred cosmetic agents according to the invention here are characterized in that they contain—based on their weight—0.001 to 2.5 wt. %, preferably 0.0025 to 1 wt. %, more preferably 0.005 to 0.5 wt. % and in particular 0.01 to 0.1 wt. % purine(s) and/or purine derivative(s). Preferred cosmetic agents according to the invention are characterized in that they contain purine, adenine, guanine, uric acid, hypoxanthine, 6-purinethiol, 6-thioguanine, xanthine, caffeine, theobromine or theophylline. In hair cosmetic preparations, caffeine is most preferred.

In another preferred embodiment of the present invention, the cosmetic agent contains ectoine ((S)-2-methyl-1,4,5,6-tetrahydro-4-pyrimidine carboxylic acid).

More preferred according to the invention are agents which contain—based on their weight—0.00001 to 10.0 wt. %, preferably 0.0001 to 5.0 wt. % and in particular 0.001 to 3 wt. % of the active agents from the group that is made up of carnitine, coenzyme Q-10, ectoine, a vitamin from the B series, a purine and derivatives or physiologically acceptable salts thereof.

A particularly preferred care additive in the hair treatment agents according to the invention is taurine. Taurine is understood to be exclusively 2-aminoethanesulfonic acid and a derivative to be the explicitly mentioned derivatives of taurine. The derivatives of taurine are understood to be N-monomethyltaurine, N,N-dimethyltaurine, taurine lysylate, taurine tartrate, taurine ornithate, lysyl taurine and ornithyl taurine.

More preferred are agents according to the invention which contain—based on their weight—0.0001 to 10.0 wt. %, preferably 0.0005 to 5.0 wt. %, more preferably 0.001 to 2.0 wt. % and in particular 0.001 to 1.0 wt. % taurine and/or a derivative of taurine.

The action of the compositions according to the invention can furthermore be increased by a 2-pyrrolidinone-5-carboxylic acid and derivatives thereof (J). Preferred are the sodium, potassium, calcium, magnesium or ammonium salts, in which the ammonium ion carries one to three C₁ to C₄ alkyl groups besides hydrogen. The sodium salt is particularly preferred. The quantities employed in the agents according to the invention are 0.05 to 10 wt. %, based on the total agent, particularly preferably 0.1 to 5 and in particular 0.1 to 3 wt. %.

Through the use of plant extracts as care substances, the hair treatment agents according to the invention can be formulated to be particularly close to nature and yet very effective in their care performance. It may even be possible in this case to omit otherwise conventional preservatives. According to the invention, primarily the extracts of green tea, oak bark, nettles, hamamelis, hops, henna, chamomile, burdock root, horsetail, hawthorn, lime blossom, almond, aloe vera, fir needle, horse chestnut, sandalwood, juniper, coconut, mango, apricot, lime, wheat, kiwi, melon, orange, grapefruit, sage, rosemary, birch, mallow, valerian, lady's smock, wild thyme, yarrow, thyme, melissa, rest harrow, coltsfoot, marshmallow, meristem, ginseng, coffee, cocoa, moringa, ginger root and ayurvedic plant extracts, such as e.g. Aegle mannelos (Bilva), Cyperus rotundus (Nagarmotha), Emblica officinalis (Amalaki), Morida citrifolia (Ashyuka), Tinospora cordifolia (Guduchi), Santalum album (Chandana), Crocus sativus (Kumkuma), Cinnamonum zeylanicum and Nelumbo nucifera (Kamala), graminaceous plants, such as wheat, barley, rye, oats, spelt, maize, the various types of millet (proso millet, finger millet, foxtail millet as examples), sugarcane, perennial ryegrass, meadow foxtail, tall oatgrass, bentgrass, meadow fescue, purple moor grass, bamboo, cottongrass, pennisetums, Andropogonodeae (Imperata cylindrica, also known as blady grass or cogon grass), buffalo grass, cord-grasses, dog's tooth grasses, lovegrasses, Cymbopogon (lemongrass), Oryzeae (rice), Zizania (wild rice), beach grass, blue oat grass, soft grasses, quaking grasses, meadow grasses, wheatgrasses and echinacea, in particular Echinacea purpurea (L.) Moench, all types of vine and pericarp of Litchi chinensis are preferred.

The plant extracts can be employed according to the invention both in pure form and in dilute form. Where they are employed in dilute form, they usually contain approx. 2-80 wt. % active agent and, as solvent, the extracting agent or mixture of extracting agents used to obtain them.

It may occasionally be necessary to use anionic polymers. Examples of anionic monomers from which these polymers can consist are acrylic acid, methacrylic acid, crotonic acid, maleic anhydride and 2-acrylamido-2-methylpropanesulfonic acid. The acid groups here can be completely or partially present as a sodium, potassium, ammonium, mono- or triethanolammonium salt. Preferred monomers are 2-acrylamido-2-methylpropanesulfonic acid and acrylic acid.

Anionic polymers containing 2-acrylamido-2-methylpropanesulfonic acid as sole monomer or comonomer, wherein the sulfonic acid group can be completely or partially present as a sodium, potassium, ammonium, mono- or triethanolammonium salt, have proved particularly effective.

More preferred is the homopolymer of 2-acrylamido-2-methylpropanesulfonic acid, which is commercially available e.g. with the name Rheothik® 11-80.

Preferred non-ionogenic monomers are acrylamide, methacrylamide, acrylic acid esters, methacrylic acid esters, vinylpyrrolidone, vinyl ethers and vinyl esters.

Preferred anionic copolymers are acrylic acid-acrylamide copolymers and in particular polyacrylamide copolymers with monomers that contain sulfonic acid groups. A polymer of this type is contained in the commercial product Sepigel® 305 from SEPPIC.

Likewise preferred anionic homopolymers are uncrosslinked and crosslinked polyacrylic acids. Preferred crosslinking agents in this case can be allyl ethers of pentaerythritol, of sucrose and of propylene. Compounds of this type are commercially available e.g. with the trademark Carbopol®.

Copolymers of maleic anhydride and methyl vinyl ether, in particular those with crosslinkages, are also color-retaining polymers. A maleic acid-methyl vinyl ether copolymer crosslinked with 1,9-decadiene is commercially available with the name Stabileze® QM.

The anionic polymers are contained in the agents according to the invention preferably in quantities of 0.05 to 10 wt. %, based on the total agent. Quantities of 0.1 to 5 wt. % are particularly preferred.

In a further embodiment, the agents according to the invention can contain non-ionogenic polymers.

Suitable non-ionogenic polymers are e.g.:
vinylpyrrolidone/vinyl ester copolymers, as marketed e.g. with the trademark Luviskol® (BASF). Luviskol® VA 64 and Luviskol® VA 73, both of them vinylpyrrolidone/vinyl acetate copolymers, are likewise preferred nonionic polymers;
cellulose ethers, such as hydroxypropyl cellulose, hydroxyethyl cellulose and methyl hydroxypropyl cellulose, as marketed e.g. with the trademarks Culminal® and Benecel® (AQUALON) and Natrosol® grades (Hercules);
starch and derivatives thereof, in particular starch ethers, e.g. Structure® XL (National Starch), a multifunctional, salt-tolerant starch;
shellac;
polyvinylpyrrolidones, as marketed e.g. with the name Luviskol® (BASF).

The nonionic polymers are contained in the compositions according to the invention preferably in quantities of 0.05 to 10 wt. %, based on the total agent. Quantities of 0.1 to 5 wt. % are more preferred.

In another embodiment, the agents according to the invention can additionally contain at least one UV light protective filter. UVB filters can be oil-soluble or water-soluble.

As oil-soluble substances, e.g. the following should be mentioned:
3-benzylidene camphor, e.g. 3-(4-methylbenzylidene) camphor;
4-aminobenzoic acid derivatives, preferably 4-(dimethylamino)benzoic acid 2-ethylhexyl ester, 4-(dimethylamino)benzoic acid 2-octyl ester and 4-(dimethylamino)benzoic acid amyl ester;
esters of cinnamic acid, preferably 4-methoxycinnamic acid 2-ethylhexyl ester, 4-methoxycinnamic acid propyl ester, 4-methoxycinnamic acid isoamyl ester, 2-cyano-3-phenylcinnamic acid 2-ethylhexyl ester (octocrylene);
esters of salicylic acid, preferably salicylic acid 2-ethylhexyl ester, salicylic acid 4-isopropylbenzyl ester, salicylic acid homomenthyl ester;
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
esters of benzalmalonic acid, preferably 4-methoxybenzmalonic acid di-2-ethylhexyl ester;
triazone derivatives, such as e.g. 2,4,6-trianilino-(p-carbo-2′-ethyl-1′-hexyloxy)-1,3,5-triazine and octyl triazone;
propane-1,3-diones, such as e.g. 1-(4-tert.-butylphenyl)-3-(4′-methoxyphenyl)propane-1,3-dione.

The following are suitable as water-soluble substances:
2-phenylbenzimidazole-5-sulfonic acid and alkali, alkaline earth, ammonium, alkylammonium, alkanolammonium and glucammonium salts thereof;
sulfonic acid derivatives of benzophenones, preferably 2-hydroxy-4-methoxybenzophenone-5-sulfonic acid and salts thereof;
sulfonic acid derivatives of 3-benzylidene camphor, such as e.g. 4-(2-oxo-3-bornylidenemethyl)benzenesulfonic acid and 2-methyl-5-(2-oxo-3-bornylidene)sulfonic acid and salts thereof.

As typical UV-A filters, derivatives of benzoyl methane are particularly suitable, such as e.g. 1-(4′-tert.-butylphenyl)-3-(4′-methoxyphenyl)propane-1,3-dione or 1-phenyl-3-(4′-isopropylphenyl)propane-1,3-dione. The UV-A and UV-B filters can, of course, also be used in mixtures. Besides the aforementioned soluble substances, insoluble pigments are also suitable for this purpose, in particular finely dispersed metal oxides or salts, such as e.g. titanium dioxide, zinc oxide, iron oxide, aluminum oxide, cerium oxide, zirconium oxide, silicates (talc), barium sulfate and zinc stearate. The particles in this case can have an average diameter of less than 100 nm, preferably between 5 and 50 nm and in particular between 15 and 30 nm They can have a spherical shape, but those particles that possess an ellipsoid shape or a shape deviating from the spherical form in another way can also be employed.

Furthermore, the cosmetic agents can contain additional active agents, auxiliary substances and additives, such as e.g.
structurants, such as maleic acid and lactic acid,
swelling agents, such as urea, allantoin, carbonates or hydantoin,
dimethyl isosorbide and cyclodextrins,
dyes for coloring the agent,
active anti-dandruff agents, such as piroctone olamine, zinc omadine and climbazole,
chelating agents, such as EDTA, NTA, β-alanine diacetic acid and phosphonic acids,
opacifiers, such as latex, styrene/PVP and styrene/acrylamide copolymers,
pearlescent agents, such as ethylene glycol mono- and distearate and PEG-3 distearate,
pigments,
stabilizers for hydrogen peroxide and other oxidizing agents,
propellants, such as propane-butane mixtures, N₂O, dimethyl ether, CO₂ and air,
antioxidants,
perfume oils, scents and fragrances.

With regard to further optional components and the quantities of these components employed, explicit reference is made to the relevant handbooks known to the person skilled in the art.

The invention therefore secondly provides a method for hair treatment in which a hair treatment agent according to claim 1 is applied onto the hair and rinsed off the hair after a period of exposure.

The period of exposure is preferably a few seconds to 100 minutes, more preferably 1 to 50 minutes and particularly preferably 1 to 30 minutes.

A method in which a cosmetic agent according to claim 1 is applied onto the hair and remains there is also in accordance with the invention. “Remains on the hair” is understood according to the invention to mean that the agent is not rinsed out of the hair immediately after its application. Instead, in this case the agent remains on the hair for more than 100 minutes up to the next hair wash.

The following examples are intended to explain the subject-matter of the present invention but without limiting it.

### Examples

Unless otherwise specified, all quantitative data are parts by weight. The following formulations were prepared using known methods of production.

Hair Rinse:

| | S1 | S2 | S3 | S4 | S5 | S6 |
|---|---|---|---|---|---|---|
| Stenol ® 1618 | 3.0 | 3.0 | 3.0 | 3.0 | 3.0 | 3.0 |
| Genamin ® KDMP | 2.0 | — | 2.0 | — | — | — |
| Cetrimonium chloride | — | — | — | — | 1.0 | — |
| Rheocare ® Ultragel | 2.0 | — | — | — | — | — |
| Dehyquart ® L80 | 0.5 | — | — | 0.5 | — | — |
| Polyquaternium-77 | 0.5 | — | — | 0.5 | — | — |
| Silcare ® SEA | 0.5 | — | 0.5 | — | — | — |
| Polyquaternium-71 | 0.5 | — | 0.5 | | — | — |
| Terraquat ® BD | — | 3.0 | 0.5 | — | 3.0 | 1.0 |
| Dow Corning ® 949 | — | — | — | 0.5 | — | — |
| Stearyldimoniumhydroxypropyl | 0.5 | — | 0.5 | 0.5 | — | — |
| laurylglucoside | | | | | | |
| Bis-ethyl(isostearylimidazoline) | 1.0 | 1.0 | 1.0 | 1.0 | 1.0 | 1.0 |
| isostearamide | | | | | | |
| Panthenol | 0.5 | 0.5 | 0.3 | 0.5 | 0.2 | 0.2 |
| Isopropyl myristate | 0.4 | — | — | — | — | — |
| DC ® 200, 60,000 cSt | 0.3 | — | 0.2 | — | — | — |
| Dicaprylyl carbonate | 0.3 | — | 0.3 | 0.3 | — | — |
| Lactic acid | 0.1 | — | — | — | 0.2 | 0.5 |
| Citric acid | — | — | — | 0.3 | — | — |
| Vanillic acid | 0.2 | 0.2 | — | 0.2 | — | — |
| Mandelic acid | — | — | 0.3 | 0.2 | 0.3 | — |
| Methylparaben | 0.2 | 0.2 | 0.2 | 0.2 | 0.2 | 0.2 |
| Fragrance | 0.3 | 0.3 | 0.3 | 0.3 | 0.3 | 0.3 |
| Phenoxyethanol | 0.4 | 0.4 | 0.4 | 0.4 | 0.4 | 0.4 |
| Water | to | to | to | to | to | to |
| | 100 | 100 | 100 | 100 | 100 | 100 |

The pH values of all of the formulations were adjusted to 2 to 4.

Deep Conditioner:

| | K1 | K2 | K3 | K4 | K5 | K6 |
|---|---|---|---|---|---|---|
| Stenol ® 1618 | 6.0 | 6.0 | 6.0 | 6.0 | 6.0 | 6.0 |
| Quartamin ® BTC 131 | 1.5 | — | 0.8 | 1.0 | — | — |
| Crodazosoft ® DBQ | 1.5 | — | — | 1.5 | — | — |
| Rheocare ® Ultragel | 3.0 | — | — | — | — | — |
| Dehyquart ® L80 | 0.5 | — | — | 0.5 | — | — |
| Dehyquart ® F 75 | — | — | — | — | 1.0 | — |
| Polyquaternium-77 | 0.5 | — | — | 0.5 | — | — |
| Silcare ® SEA | 0.5 | 0.5 | 0.5 | — | — | — |
| Polyquaternium-71 | 0.5 | — | 0.5 | — | — | — |
| Terraquat ® BD | — | 4.5 | 0.5 | 1.5 | 4.5 | 1.5 |
| Dow Corning ® 949 | — | — | — | 0.5 | — | — |
| Stearyldimoniumhydroxypropyl | 0.5 | — | 0.5 | 0.5 | — | — |
| laurylglucoside | | | | | | |
| Bis-ethyl(isostearylimidazoline) | 1.0 | 1.5 | 4.0 | 3.0 | 1.5 | 1.5 |
| isostearamide | | | | | | |
| Lactic acid | 0.5 | — | — | — | 2.0 | — |
| Citric acid | — | — | — | 0.3 | — | 3.0 |
| Vanillic acid | 2.0 | 2.0 | — | 0.5 | — | — |
| Mandelic acid | — | — | 0.3 | 2.0 | 0.3 | — |
| Isopropyl myristate | 0.4 | — | 0.4 | — | — | — |
| Panthenol | 0.5 | 0.5 | 0.8 | 0.5 | 0.5 | 0.5 |
| DC ® 200, 60,000 cSt | 0.3 | — | 0.2 | — | — | — |
| Cetiol ® C5 | 1.0 | — | 0.3 | 0.3 | — | — |
| Dicaprylyl carbonate | 0.3 | — | 0.3 | 0.3 | — | — |
| Methylparaben | 0.2 | — | 0.2 | 0.2 | 0.2 | 0.2 |
| Fragrance | 0.3 | 0.3 | 0.3 | 0.3 | 0.3 | 0.3 |
| Phenoxyethanol | 0.4 | 0.4 | 0.4 | 0.4 | 0.4 | 0.4 |
| Water | to | to | to | to | to | to |
| | 100 | 100 | 100 | 100 | 100 | 100 |

The pH values of all of the formulations were adjusted to 2 to 4.

Hair Shampoo:

| | HS1 | HS2 | HS3 | HS4 | HS5 |
|---|---|---|---|---|---|
| Ammonium lauryl ether sulfate (2 EO) | 20.0 | 20.0 | 9.0 | — | — |
| Sodium lauryl ether sulfate (2 EO) | — | — | — | 9.0 | 9.0 |
| Terraquat ® BD | 2.0 | 2.0 | — | — | — |
| Ammonium lauryl sulfate (30%) | 20.0 | 20.0 | — | — | — |
| Cocamidopropyl betaine | 7.0 | 7.0 | 3.0 | 3.0 | 3.0 |
| Plantacare ® 818 UP | — | — | 6.0 | 6.0 | 6.0 |
| Sodium benzoate | 0.2 | 0.2 | 0.2 | 0.2 | 0.2 |
| Citric acid or lactic acid or | 1.0 | 1.0 | 1.0 | 1.0 | 1.0 |
| a mixture of both | | | | | |
| Thickening agent | 1.0 | 2.0 | 0.5 | 2.5 | 1.0 |
| Polyquaternium-77 | 0.5 | — | — | 0.5 | — |
| Silcare ® SEA | 0.5 | — | 0.5 | — | 0.3 |
| Cosmedia ® Guar C261 | — | — | — | 0.4 | — |
| Polyquaternium-71 | 0.5 | — | 0.5 | — | 0.1 |
| Polyquaternium-74 | — | — | 0.3 | — | 0.2 |
| Dow Corning ® 949 | — | — | — | 0.5 | — |
| Stearyldimoniumhydroxypropyl | 0.5 | — | 0.5 | 0.5 | 0.5 |
| laurylglucoside | | | | | |
| Bisethyl(isostearylimidazoline)isostearamide | 2.0 | 2.0 | 3.0 | 4.0 | 1.0 |
| Lactic acid | 0.5 | — | — | — | 2.0 |
| Citric acid | — | — | — | 0.3 | — |
| Vanillic acid | 2.0 | 2.0 | — | 0.5 | — |
| Mandelic acid | — | — | 0.3 | 2.0 | 0.3 |
| Isopropyl myristate | 0.4 | — | 0.4 | — | — |
| Panthenol | 0.5 | 0.5 | 0.8 | 0.5 | 0.5 |
| DC ® 200, 60,000 cSt | 0.3 | — | 0.2 | — | — |
| Cetiol ® C5 | 1.0 | — | 0.3 | 0.3 | — |
| Dicaprylyl carbonate | 0.3 | — | 0.3 | 0.3 | — |
| Fragrance | 0.3 | 0.3 | 0.3 | 0.3 | 0.3 |
| Phenoxyethanol | 0.4 | 0.4 | 0.4 | 0.4 | 0.4 |
| Water | to 100 | to 100 | to 100 | to 100 | to 100 |

The pH values of all of the formulations were adjusted to 4.5 to 5.8.

As thickener it is possible to use any thickeners or thickener systems known to the person skilled in the art in surfactant systems. For example, cellulose ethers, xanthan gums, hydroxyethyl celluloses, laureth-2 and laureth-3, as well as e.g. the products with the trade name Antil® or Crothix®, can be employed particularly advantageously in the above-mentioned formulations, each individually or in mixtures with one another.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A hair treatment agent, comprising—based on its weight—
a) 0.01 to 15 wt. % of a fatty acid amide according to formula (I) 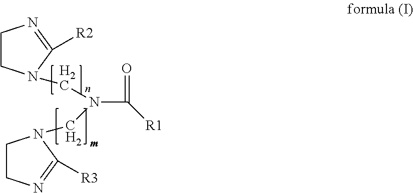
in which R1, R2 and R3 independently of one another denote a linear branched or unbranched C6 to C30, preferably C8 to C24, more preferably C12 to C22 and most preferably C12 to C18 alkyl or alkenyl group, wherein furthermore R2 preferably equals R3 and most preferably R1 equals R2 equals R3, and n and m independently of one another denote integers from 1 to 10, preferably 2 to 6 and most preferably 2, 3 and/or 4, wherein it is most preferred that n=m, and
b) 0.001 to 5.0 wt. % of at least one fragrance.

2. The hair treatment agent according to claim 1, further comprising 0.1 to 15.0 wt. % of at least one quaternary ammonium compound selected from at least one of the group consisting of:
the cationic surfactants of the formula (Tkat1) 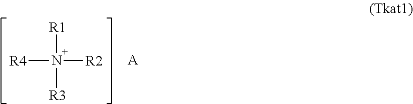
in which R1, R2, R3 and R4 each independently of one another denote hydrogen, a methyl group, a phenyl group, a benzyl group, a saturated, branched or unbranched alkyl residue with a chain length of 8 to 30 carbon atoms, which may optionally be substituted with one or more hydroxy groups, and A denotes a physiologically acceptable anion, e.g. halides such as chloride or bromide as well as methosulfates;
esterquats; or
a compound of the general formula (bI) 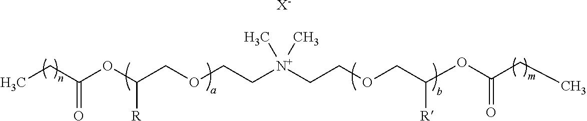
formula (bI), in which
n and m independently of one another denote integers between 5 and 40, with the proviso that n+m≧38; more preferably n=m; most preferably n=m=20;
a and b independently of one another denote integers between 1 and 10; independently of one another they particularly denote 1, 2, 3, 4 or 5; the equation a+2≧b≧a−2 preferably applies here and most preferably a=b=3;
R and R′ are selected independently of one another from —H and —CH₃; R═R′ preferably applies, so that preferably either PEG or PPG diesterquats are employed; R═R′=—CH3 particularly preferably applies;
X⁻ is a physiologically acceptable anion, a halide such as chloride, bromide or iodide, toluenesulfonate, methosulfate etc., and is more preferably methosulfate;
a compound of the formula (bII) 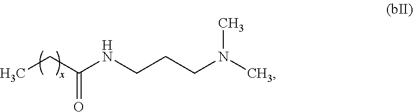
in which x denotes 18, 19, 20, 21, 22, 23 or 24;
the quaternary imidazolines of the formula (bIII), 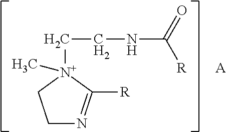
in which the residues R each independently of one another denote a saturated or unsaturated, linear or branched hydrocarbon residue with a chain length of 8 to 30 carbon atoms and A denotes a physiologically acceptable anion,
poly(methacryloyloxyethyltrimethylammonium compounds);
polyquaternium-24;
polyquaternium-67;
polyquaternium-72;
cationized honey;
polymeric dimethyldiallylammonium salts and copolymers thereof with esters and amides of acrylic acid and methacrylic acid;
copolymers of vinylpyrrolidone with quaternized derivatives of dialkylaminoalkyl acrylate and methacrylate;
vinylpyrrolidone/vinylimidazolium methochloride copolymers and/or
quaternized polyvinyl alcohol;
polyquaternium 2;
polyquaternium-7;
polyquaternium-16;
polyquaternium 17;
polyquaternium 18;
polyquaternium 27;
polyquaternium-69;
polyquaternium-74;
polymeric alkyl oligoglucoside of the formula 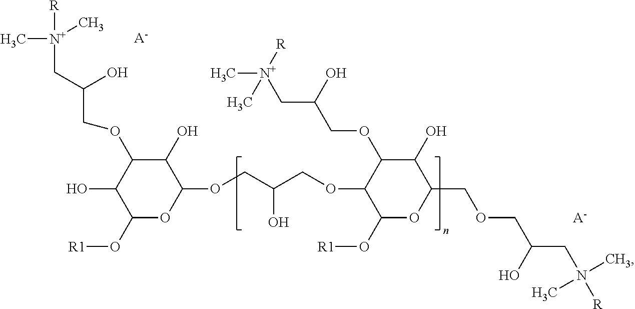
in which R denotes a linear or branched C6 to C30 alkyl residue, a linear or branched C6 to C30 alkenyl residue, R2 denotes a linear or branched C6 to C30 alkyl residue, a linear or branched C6-C30 alkenyl residue and A⁻ denotes a physiologically acceptable anion;
oligomeric alkyl oligoglucoside of the formula 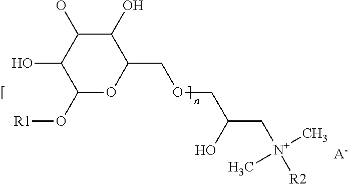
in which R1 denotes a linear or branched C6 to C30 alkyl residue, a linear or branched C6 to C30 alkenyl residue, R2 denotes a linear or branched C6 to C30 alkyl residue, a linear or branched C6-C30 alkenyl residue and A⁻ denotes a physiologically acceptable anion; and
polyquaternium-71.

3. The hair treatment agent according to claim 1, further comprising one hydroxycarboxylic acid.

4. The hair treatment agent according to claim 1, further comprising at least one silicone compound.

5. The hair treatment agent according to claim 4, wherein the silicone includes an amino functional silicone.

6. The hair treatment agent according to claim 1, further comprising carnitine.

7. The hair treatment agent according to claim 1, further comprising a purine compound.

8. The hair treatment agent according to claim 7, wherein the purine compound is caffeine.

9. The hair treatment agent according to claim 1, further comprising ectoine.

10. The hair treatment agent according to claim 1, further comprising at least one ubiquinone.

11. The hair treatment agent according to claim 1, wherein the ubiquinone includes coenzyme Q-10.

12. A method of treating keratinic fibers, comprising applying onto the keratinic fibers a hair treatment agent as set forth in claim 1; and
after an exposure time of a few seconds to 45 minutes, rinsing the hair treatment agent from the hair.
